(19) United States
(12) Patent Application Publication
(10) Pub. No.: US 2024/0017061 A1
(43) Pub. Date: Jan. 18, 2024

(54) Handheld Electroporation Devices, And Related Systems And Methods

(71) Applicant: Inovio Pharmaceuticals, Inc., Plymouth Meeting, PA (US)

(72) Inventors: Beat Stadelmann, San Diego, CA (US); Andrew Booth, La Jolla, CA (US); Jay McCoy, Temecula, CA (US); Charu Mather, San Diego, CA (US)

(21) Appl. No.: 18/174,298

(22) Filed: Feb. 24, 2023

Related U.S. Application Data
(60) Provisional application No. 63/314,282, filed on Feb. 25, 2022.

(51) Int. Cl. A61N 1/32; H02J 7/34; H02J 7/00

(52) U.S. Cl. CPC A61N 1/327; H02J 7/345; H02J 7/0047; H02J 7/00712; H02J 7/0013

(57) ABSTRACT

A method for preparing an electroporation device to deliver an electroporation treatment includes steps of delivering a charge current from at least one battery through a charge circuit to a supercapacitor unit and charging the supercapacitor unit with the charge current. The charging step includes measuring one or more input parameters of the charge current while the charge current is in at least one charge state of a plurality of charge states of charging the supercapacitor unit. The charging step also includes at least one step of transitioning the charge current between charge states of the plurality of charge states responsive to the one or more measured input parameters. Transitioning the charge current includes adjusting a magnitude of the charge current. In this method, the measuring and transitioning steps are automatically controlled by a control unit executing machine-readable instructions.

### CROSS REFERENCE TO RELATED APPLICATIONS

The present application claims the benefit of U.S. Provisional Application No. 63/314,282, filed Feb. 25, 2022, in the name of Stadelmann et al., the entire contents of which are incorporated herein by this reference.

### TECHNICAL FIELD

The present invention relates to electroporation, and more particularly to electroporation devices for increasing in vivo transfection of injected agents.

### BACKGROUND

The classical mode of administering vaccines and other pharmaceutical agents into the body tissues is by direct injection into muscle or skin tissues using a syringe and needle. Incorporating electroporative pulses of electric energy at or near the injection site is known to facilitate delivery of such vaccines or agents directly into the cells within the tissue. Such direct delivery to cells using electroporative electric pulses can have a profound clinical effect on the quality of the response of the body's metabolic and/or immune systems over that of simple syringe and needle injection. Moreover, the capability of direct delivery of agents into the cell via electroporation has enabled the effective delivery of therapeutic agents (e.g., DNA-encoded monoclonal antibodies (dMAb), expressible naked DNA encoding a polypeptide, expressible naked DNA encoding a protein, recombinant nucleic acid sequence encoding an antibody, and the like) having any number of functions, including antigenic for eliciting of immune responses, or alternatively, metabolic for affecting various biologic pathways that result in a clinical effect.

Handheld electroporation devices typically require either wired connection to a power source or a bulky battery assembly to provide sufficient power delivery for multiple in vivo electroporation treatments (i.e., for numerous patients). Such electroporation devices are less effective for remote treatment settings, such as remote areas and/or in less-developed countries, where power grids might lack reliability or may require uncommon electric outlet (e.g., power plug and socket) configurations and where various battery types may be unavailable. Such electroporation devices are also less effective for mass treatment circumstances, such as mass vaccinations and/or immunization circumstances, in which treatments are needed to serve large populations of at-risk individuals. Such electroporation devices are also less effective for long-term storage and/or stockpiling scenarios, where the devices may need to be stored for potentially long durations and retrieved promptly to provide reliable treatments. Internal permanent batteries, which are often used in battery-powered handheld electroporation devices, have a limited shelf life and are typically not an option for stockpiling or long-term storage. The ongoing coronavirus pandemic has demonstrated the stark need for mass-producible, user-friendly, battery powered handheld treatment devices that work promptly, reliably, and efficiently across high quantities of uses and potentially after long-term storage.

### SUMMARY

According to an embodiment of the present disclosure, a method for preparing an electroporation device for delivering an electroporation treatment includes steps of delivering a charge current from at least one battery through a charge circuit to a supercapacitor unit and charging the supercapacitor unit with the charge current. The charging step includes measuring one or more input parameters of the charge current while the charge current is in at least one charge state of a plurality of charge states of charging the supercapacitor unit. The charging step also includes at least one step of transitioning the charge current between charge states of the plurality of charge states responsive to the one or more measured input parameters. Transitioning the charge current includes adjusting a magnitude of the charge current. In this method, the measuring and transitioning steps are automatically controlled by a control unit executing machine-readable instructions.

According to another embodiment of the present disclosure, a method of using a handheld electroporation device includes a step of delivering a charge current from at least one battery through a charge circuit to a supercapacitor unit, wherein the charge circuit and the supercapacitor unit are disposed within a device housing, and a step of charging the supercapacitor unit with the charge current. The charging step includes measuring at least one voltage parameter of the at least one battery and adjusting a magnitude of the charge current to a current magnitude associated with a primary charge state responsive to the at least one measured voltage parameter. The measuring and transitioning steps are automatically controlled by a control unit executing machine-readable instructions. The method also includes steps of discharging an output signal from the supercapacitor unit after the supercapacitor unit is fully charged, converting the output signal to one or more electroporation pulses, and transmitting the one or more electroporation pulses to at least one electrode of the handheld electroporation device.

According to an additional embodiment of the present disclosure, a power supply unit for an electroporation device includes a battery unit, which is configured to connect interchangeably to a first type of battery and a second type of battery, and an energy storage unit that is configured to be charged interchangeably by the first type of battery and the second type of battery and to discharge a total energy of at least about 30 Joules. The power supply unit includes an integrated circuit configured to execute computer readable instructions and a charge circuit that is in electrical communication with the battery unit and the energy storage unit. The charge circuit is operable under control of the integrated circuit such that: the charge circuit is configured to measure one or more input parameters of a charge current drawn from the battery unit and is further configured to adjust a magnitude of the charge current responsive to the measured one or more input parameters while charging the energy storage unit with the charge current.

According to a further embodiment of the present disclosure, a handheld electroporation device includes a device housing, at least one electrode connectable thereto, and a battery unit that is at least partially insertable within the device housing and is configured to connect interchangeably to a first type of battery and a second type of battery. The device includes a supercapacitor unit that is configured to be charged interchangeably by the first type of battery and the second type of battery and is further configured to discharge a total energy of at least about 30 Joules. A charge circuit is in electrical communication with the battery unit and the supercapacitor unit and is configured to deliver a charge current from the battery unit to the supercapacitor unit for charging the supercapacitor unit. The charge circuit, operating under control of an integrated circuit configured to execute machine readable instructions, is configured to measure at least one voltage parameter of the respective first type of battery or second type of battery and to responsively adjust a magnitude of charge current to thereby reduce a rate at which the respective first type of battery or second type of battery loses charge while charging the supercapacitor unit.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a perspective view of a handheld electroporation device, according to an embodiment of the present disclosure;

FIG. 1B is another perspective view of the handheld electroporation device of FIG. 1A taken at an opposite perspective;

FIG. 2A is a partially exploded perspective view of a distal portion of the electroporation device illustrated in FIG. 1A, showing an electrode array attachable to a distal portion of a housing member of the device;

FIG. 2B is a side plan view of a distal portion of the electroporation device illustrated in FIG. 1A;

FIG. 2C is a diagram view of a physician using the electroporation device illustrated in FIG. 1A to treat a subject, according to an embodiment of the present disclosure;

FIG. 3A is a perspective view of an electrode array assembly that includes the electrode array illustrated in FIG. 2A and a safety cap coupled therewith;

FIGS. 3B and 3C are perspective and top plan views, respectively, of the electrode array assembly illustrated in FIG. 3A disposed in a sterile packaging container, according to an embodiment of the present disclosure;

FIG. 3D is a perspective view of a bulk packaging assembly that includes a multiplicity of the sterile packaging containers illustrated in FIGS. 3B and 3C;

FIGS. 4A and 4B are opposite side plan views of the handheld electroporation device illustrated in FIG. 1A, with an exterior housing member removed to show internal components of the device;

FIG. 4C is a perspective, partially exploded view of the electroporation device illustrated in FIGS. 4A and 4B, showing features of a battery compartment of the device and batteries usable with the device, according to an embodiment of the present disclosure;

FIG. 4D is another perspective, partially exploded view of the electroporation device illustrated in FIGS. 4A and 4B, with an additional housing member removed for illustrative purposes;

FIG. 4E is an exploded view of internal components of the electroporation device illustrated in FIGS. 4A-4D, including a circuit board for connecting the battery compartment to an energy storage unit;

FIG. 5 is a diagram view of a power supply unit and constituent electrical components for use with the electroporation device illustrated in FIGS. 1A-4D;

FIG. 6 is a diagram view of an algorithm for charging an energy storage unit of the device using a pair of batteries, according to an embodiment of the present disclosure; and

FIG. 7 is a side plan view of an electroporation system that includes a handheld electroporation device and a base station, according to an embodiment of the present disclosure.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although the terms first, second, etc. may be used herein to describe various elements, these elements should not be limited by these terms. These terms are instead used to distinguish one element from another. For example, a first element could be termed a second element, and, similarly, a second element could be termed a first element without departing from the scope of the embodiments disclosed herein.

The embodiments disclosed herein pertain to devices and methods for storing, charging, and releasing electroporative energy within relatively small, user-friendly handheld electroporation devices adapted to perform in vivo, reversible electroporation. For example, the embodiments herein include handheld electroporation devices and techniques that store energy internally in the devices at low voltage that yet produce a high, virtually instantaneous current for reversibly electroporating tissue. Additionally, the electroporation devices and techniques described herein have energy storage units that can be fully charged in short timeframes (e.g., 3 minutes or less) using common, widely available batteries, which charge the energy storage units under the control of control units that are located within the devices and are also powered by the batteries. These features further serve to conserve overall space within the handheld electroporation devices, allowing them to be made smaller and lighter compared to state of the art handheld electroporation devices, also allowing the devices herein to be mass produced at industrial scales that are particularly advantageous for mass vaccination type settings. Furthermore, the energy storage, charging, and discharging features described herein allow the electroporation devices to be stored in long-term, stockpile setting and yet be usable virtually immediately upon retrieval with widely available, off-the-shelf battery types. These features, as incorporated into the handheld electroporation devices described herein, provide small, self-contained, storable, widely usable, and mass-producible handheld electroporation devices. These features are facilitated by an algorithm that is uniquely adapted to instruct the control unit to extract substantially the maximum energy at substantially the highest efficiency from the batteries to charge the energy storage unit without negatively impacting the power supply to the control unit. Stated differently, the algorithm substantially optimizes the maximum allowable current drawn from a battery unit while maintaining an acceptable operating voltage for an electronic control circuit to operate. By adjusting the charge current to provide highly efficient charging characteristics, the algorithm also provides an added safety benefits of protecting the batteries from overstress, overheating, inefficient or insufficient output, catching fire and/or other potential damage.

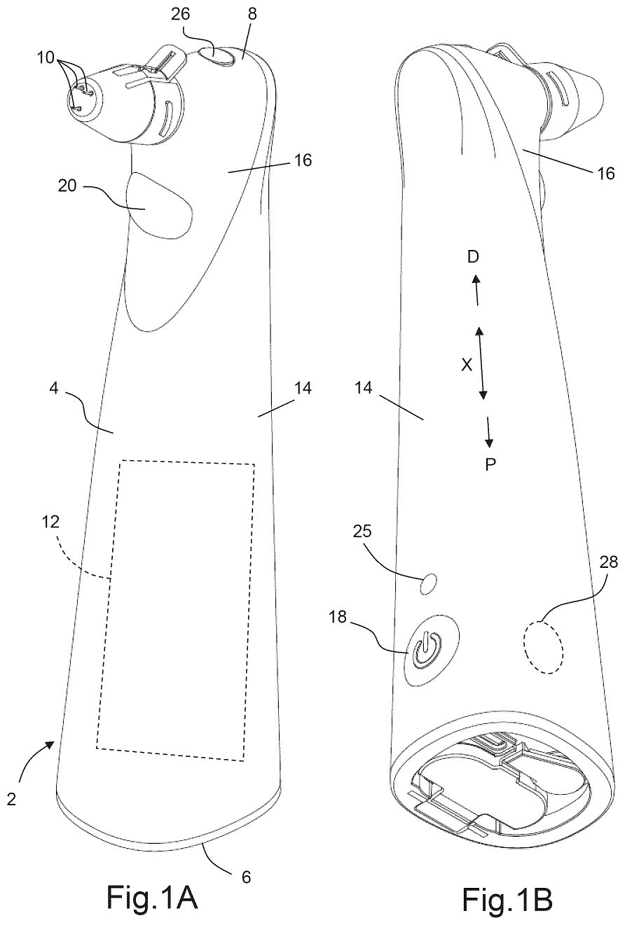

Referring to FIGS. 1A-1B, an example embodiment of a battery powered handheld electroporation device 2 is shown. The device 2 includes a handle body 4 having a proximal end 6 to a distal end 8 spaced from each other along a longitudinal direction X. The handle body 4 is configured to carry at least one electrode 10 at the distal end 8. In the illustrated embodiment, the handle body 4 is configured to carry a plurality of electrodes 10, particularly three (3) electrodes 10 arranged in a triangular pattern. In other embodiments, the handle body 4 can be configured to carry two (2) electrodes 10. In additional embodiments, the handle body 4 can be configured to carry other quantities of electrodes 10, such as four (4), five (5), six (6), seven (7), eight (8), nine (9), ten (10), eleven (11), twelve (12), or more than twelve electrodes 10. In multi-electrode 10 embodiments, the electrodes 10 can be arranged in various patterns, such as circular, polygonal, concentric, grid, and irregular patterns, by way of non-limiting examples. In other embodiments, the handle body 4 can be configured to carry a single electrode 10. Thus, although the following description refers to plural electrodes 10, it should be appreciated that the present disclosure encompasses embodiments with a single electrode 10. The electrodes 10 are configured to deliver one or more pulses of electrical energy to cells of target tissue in vivo, specifically for reversibly electroporating the cells. Any such electrical pulse can be referred to as an “electroporation pulse.” The device 2 includes a power supply unit 12 and circuitry for providing electrical communication between the power supply unit 12 and the electrodes 10 for generating and transmitting the one or more electroporation pulses to the electrodes 10, as described in more detail below.

The handle body 4 includes one or more housing members 14, 16 that define the handle body 4 and house internal components of the device 2. In the illustrated embodiment, the handle body 4 includes a first housing member 14 that extends from the proximal end 6 to the distal end 8 in a distal direction D and a second housing member 16 located at the distal end 8. The distal direction D is opposite a proximal direction P. It should be appreciated that the distal and proximal directions D, P are each mono-directional components of the longitudinal direction X, which is bi-directional. It should also be appreciated that, as used herein: the terms “longitudinal”, “longitudinally”, and derivatives thereof refer to the longitudinal direction X; the terms “distal”, “distally”, and derivatives thereof refer to the distal direction D; and the terms “proximal”, “proximally”, and derivatives thereof refer to the proximal direction P. The first housing member 14 houses the power supply unit 12 and additional internal components of the device 2. The second housing member 16 is configured for receiving the electrodes 10. The first and second housing members 14, 16 define respective openings, access ports, apertures, and the like for additional components of the device 2, such as user interface components, as described in more detail below. It should be appreciated that the first and second housing members 14, 16 can collectively be referred to as the “device housing” 14, 16. It should also be appreciated that in other embodiments the handle body 4 can employ other device housing configurations, such as a clamshell device housing configuration, a single-piece device housing configuration, or other device housing configurations.

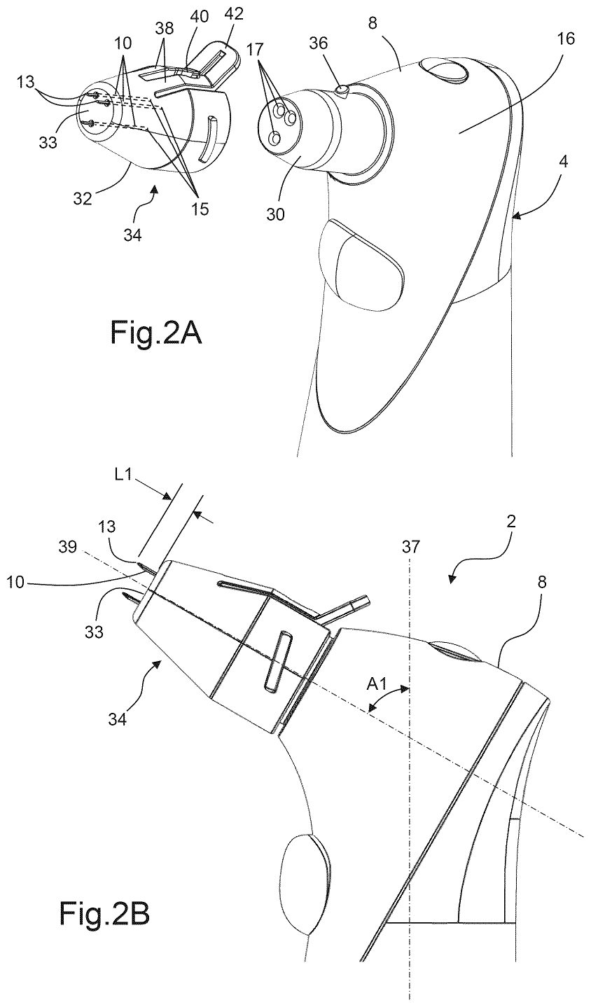

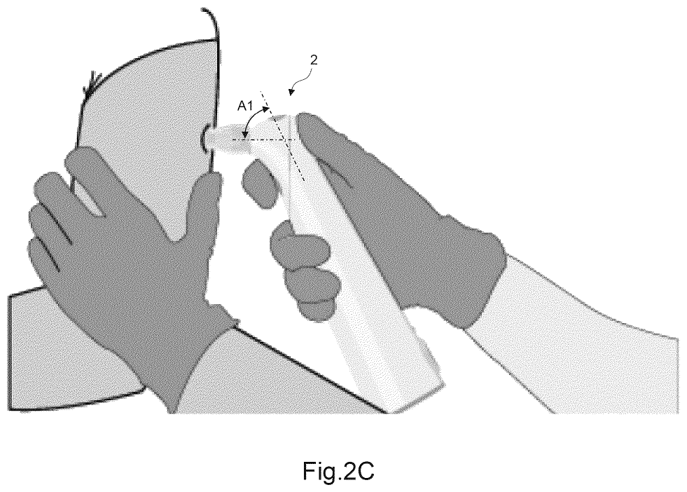

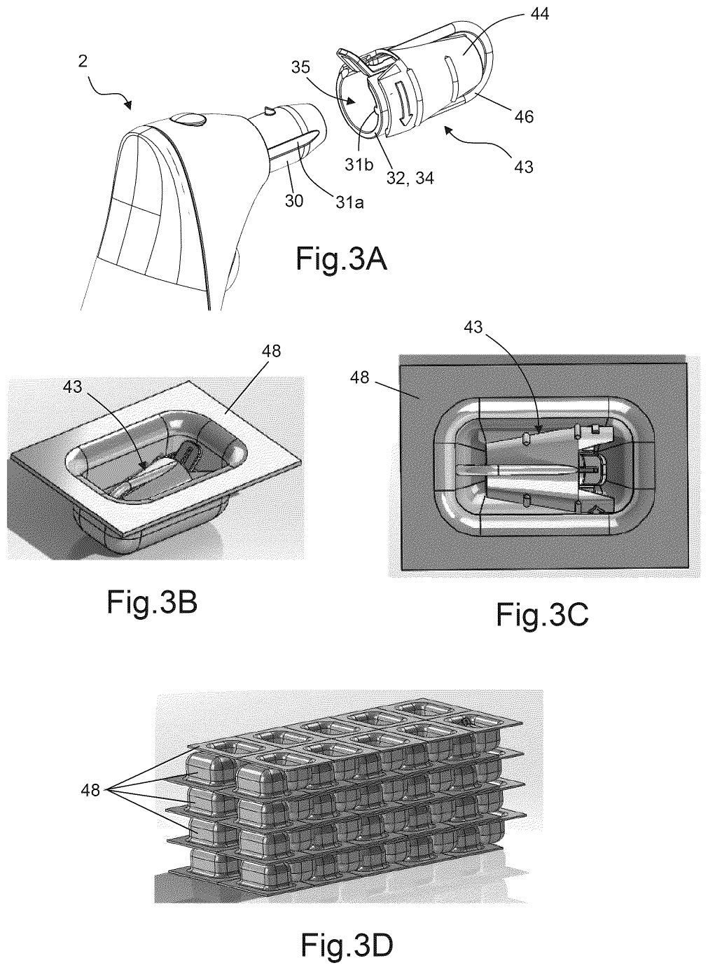

Referring now to FIGS. 2A-2B, the distal end 8 of the handle body 4 includes an electrode mount 30 for receiving the electrodes 10. In the illustrated embodiment, the electrode mount 30 is provided on the second housing member 16. The electrodes 10 are carried by an electrode applicator 32, which, together with the electrodes 10, can be referred to as an “electrode array” 34. The electrode applicator 32 carries the electrodes 10 such that distal ends 13 of the electrodes 10 extend forward from a front face 33 of the electrode applicator 32 and proximal ends 15 of the electrodes 10 extend rearwardly toward the handle member 4. The electrode array 34 and the electrode mount 30 have complimentary geometries allowing the electrode array 34 to be removably attachable to and detachable from the electrode mount 30. In particular, the electrode mount 30 is receivable within a rear receptacle 35 of the electrode applicator 32 (see FIG. 3A) for coupling therewith. The proximal ends 15 of the electrodes 10 are receivable within sockets 17 defined within the electrode mount 30 for providing electrical communication between the electrodes 10 and the circuitry of the device 2.

The electrode mount 30 and the electrode array 34 preferably include complimentary locking structures for securely attaching the electrode array 34 to the electrode mount 30. In the illustrated embodiment, the locking structures include a locking post 36 disposed on the electrode mount 30 and a complimentary locking arm 38 defined by the electrode applicator 32. The locking arm 38 defines a locking slot 40 configured to receive the locking post 36 of the electrode mount 30 and couple therewith, such as in a snap-fit fashion. In this manner, the coupled engagement of the locking post 36 with the locking arm 38 prevents unintended decoupling of the electrode applicator 32 from the electrode mount 30. The locking arm 38 includes a release tab 42, which can extend outwardly from the electrode applicator 32 and is configured to allow a user to flex the locking arm 38 outward in a manner disengaging the locking post 36 from the locking slot 40, thereby allowing the user to detach the electrode applicator 32 from the electrode mount 30 when desired, such as between uses. In this manner, the device 2 can be configured for single-use electroporation applications, whereby after each use, the physician can remove the used electrode array 34 and attach a new electrode array 34 for the next treatment subject. The used electrode arrays 34 can be discarded or sterilized for subsequent use.

As shown in FIG. 2B, the electrode applicator 32 carries the electrodes 10 at a maximum electrode depth L1 measured from the distal ends 13 of the electrodes 10 to the front face 33 of the electrode applicator 32. In the illustrated embodiment, the maximum electrode depth L1 is set for intradermal (ID) electroporation and is in a range from about 0.01 mm to about 5.0 mm. It should be appreciated that in other embodiments the electrodes 10 can be configured for intramuscular (IM) electroporation and have a maximum electrode depth L1 in a range from about 5.0 mm to about 35 mm. In yet other embodiments, the electrodes 10 can be configured for adipose electroporation and have a maximum electrode depth L1 in a range from about 4.0 mm to about 20 mm. In further embodiments, the electrodes 10 can be configured for multi-depth electroporation, such as for targeting any combination of intradermal, adipose, intramuscular, and/or mucosal tissue.

In the illustrated embodiment, the electrode mount 30 positions the electrode array 34 at an application angle A1 from a longitudinal axis 37 of the device 2, which longitudinal axis 37 is oriented along the longitudinal direction X. For purposes of this disclosure, the application angle A1 is defined as the angle that subtends: (1) from a location on the longitudinal axis 37 distally spaced from the distal end 8 of the handle body 4, (2) to a central axis 39 of the electrode array 34. The application angle A1 is in a range from about 0 degrees to about 90 degrees, and more particularly from about 45 degrees to about 80 degrees, and more particularly from about 55 degrees to about 65 degrees. As shown in FIG. 2C, the application angle A1 provides the device 2 with an ergonomic design advantageously adapted for treating subjects.

Referring now to FIG. 3A, the electrode array 34 can be disposed within a removable safety cap 44 that is configured to cover the electrodes 10 prior to treatment. The electrode array 34 and the safety cap 44 together comprise an array assembly 43. The cap 44 preferably includes one or more grip features 46 to facilitate handling by the user, such as for attaching the electrode array 34 to the mount 30. As shown, the mount 30 can include a guide member 31a, such as a guide slot 31a, which receives a complimentary guide member 31b of the applicator 32, such as a guide protrusion 31b, to facilitate proper alignment of the proximal ends 15 of the electrodes 10 with the sockets 17 of the mount 30 during applicator 32 attachment. It should be appreciated that the cap 44 and the applicator 32 of the illustrated embodiment are cooperatively connectable to each other in such a manner to prevent inadvertent decoupling from one another. The coupling force between the cap 44 and actuator 32 is configured to be readily overcome once the applicator 32 is attached to the mount 30, thereby facilitating easy cap removal when the device 2 is treatment ready.

As shown in FIGS. 3B-3C, the array assembly 43 can be provided in individual sterile packaging containers, such as the illustrated “tray” type container 48. In other embodiments, the array assembly 43 can be provided in a sterile peel pouch container, a sterile blister type container, or other types of sterile containers. As shown in FIG. 3D, the tray type containers 48 can advantageously be nested together for bulk packaging. It should be appreciated that the electrode arrays 34 are preferably configured for single-use treatment. For such single-use treatments, the physician removes the array assembly 43 from the sterile packaging container and attaches the array assembly 43 to the mount 30. For the illustrated embodiment, during attachment, the physician aligns the guide protrusion 31b of the applicator 32 with the guide slot 31a of the mount 30, which facilitates insertion of the electrode proximal ends 15 into the sockets 17. The physician presses the applicator 32 toward the mount 30 until the locking post 36 of the electrode mount 30 couples with the locking slot 40 of the locking arm 38, which coupling can be indicated by an audible and/or tactile “click” or “snap”. After the array assembly 43 is coupled to the mount 30 (and preferably once the device 2 is treatment ready), the physician can remove the safety cap 44 and administer the electroporation treatment.

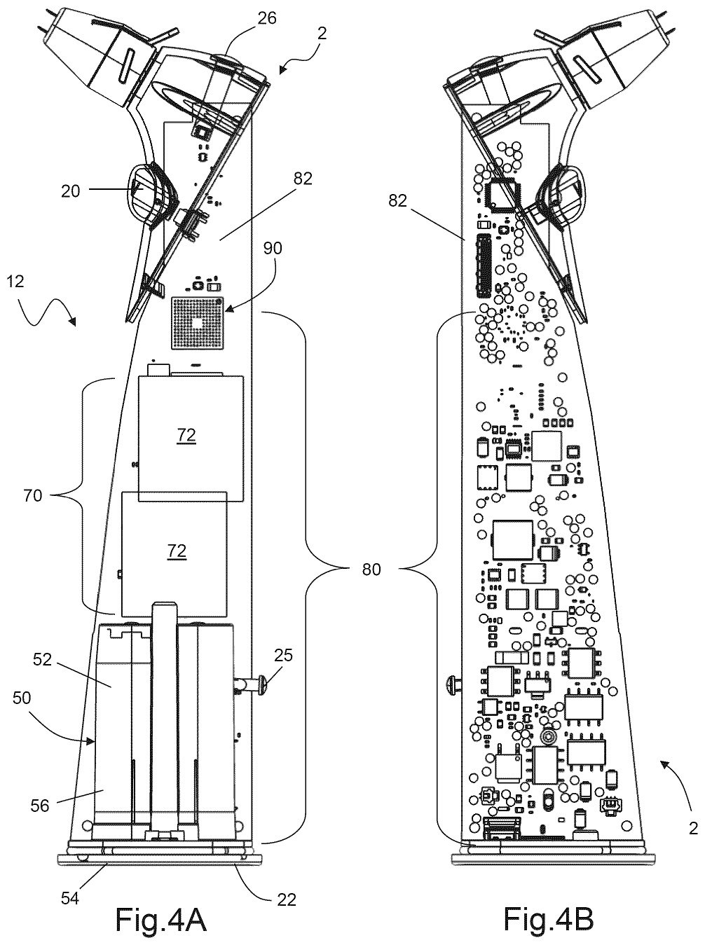

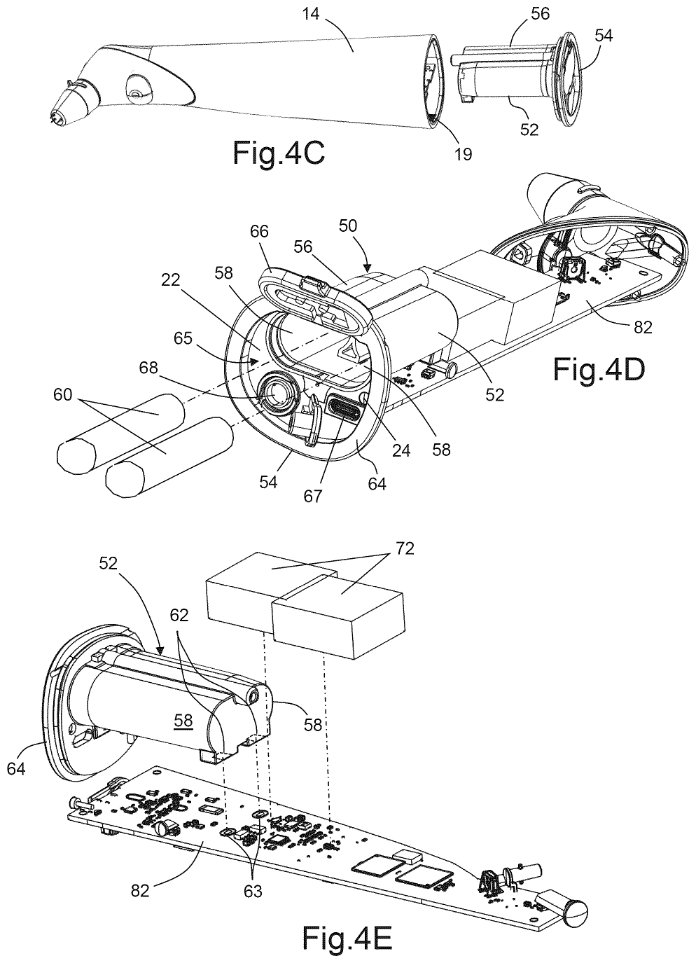

Referring now to FIGS. 4A-4E, the power supply unit 12 includes a battery unit 50, an energy storage unit 70, a charge circuit 80 that provides electrical communication between the battery unit 50 and the energy storage unit 70, and a control unit 90 configured to control operation of the charge circuit 80 for transmitting electrical charge from the battery unit 50 to charge the energy storage unit 70 with sufficient energy to deliver one or more electroporation pulses to tissue in vivo. As shown, the charge circuit 80 is disposed on a circuit board, which can be a printed circuit board (PCB) 82. The power supply unit 12 also includes user interface components, which include a power button 18 to turn the device ON and OFF, an electroporation delivery trigger or button 20, an access hub 22, and one or more indicator devices, such as LED displays 24, 25, 26 and/or speaker(s) 28 (see FIGS. 1A-1B) for indicating various status information of the device 2, as described in more detail below. For example, the indicator devices of the illustrated embodiment include a power LED display 25, which can be located adjacent the power button 18 and indicates when the power button 18 has been activated to turn the device ON. The power LED display 25 is preferably a multi-color LED display and is preferably configured to also indicate charging status information of the energy storage unit 70 (i.e., the charge status of the energy storage unit 70). The indicator devices of the illustrated embodiment also include a treatment LED display 26, which can be located adjacent the electrode mount 30 and is configured to indicate a treatment status of the device 2 (e.g., when the device 2 is at a “treatment ready” status, meaning ready to deliver one or more electroporation pulses).

The battery unit 50 includes a battery housing 52 that is receivable at least partially within the device housing 14, 16. For example, as shown in FIG. 4C, in the illustrated embodiment, the battery housing 52 includes a proximal end 54 and an insertion portion 56 that extends distally from the proximal end 54 and is partially insertable within the first housing member 14 through a proximal access opening 19 thereof. As shown in FIG. 4D, the insertion portion 56 defines one or more battery receptacles 58 each configured to hold a battery 60, as described in more detail below. As shown in FIG. 4E, each battery receptacle 58 includes an output terminal 62, which is configured to be in electrical communication with the charge circuit 80, such as via respective contacts 63 on the PCB 82. In the illustrated embodiment, the battery housing 52 includes a base support member 64, such as a ring-stand 64, which extends along a periphery of the proximal end 54. The base support member 64 is configured to allow the device 2 to free-stand upon the proximal end 54.

As shown in FIG. 4D, in the illustrated embodiment, the battery housing 52 also includes the access hub 22, which is located adjacent the proximal end 54 of the battery housing 52 and is accessible through a proximal opening 65 defined by the base support member 64. The access hub 22 includes a battery cover 66, which iterates between a closed position (FIG. 1B), in which any batteries 60 within the battery receptacles 58 are affixed in place and in contact with the output terminals 62, and an open position, in which the batteries 60 are insertable within and/or removable from the battery receptacles 58. As shown, the battery cover 66 can be a swing-latch type cover, although other cover types are within the scope of the present disclosure. The access hub 22 can also include an electronic port 67, such as a USB-C port 67, which can be configured for charging the one or more batteries 60 and/or providing electronic communication with an external computer device, such as for monitoring the operation and/or status of the device 2. The access hub 22 can include an indicator device, such as a battery-charging LED display 24 for indicating when the batteries 60 are being charged via the electronic port 67. The access hub 22 can also include an additional port 68, which can provide access to the interior of the first housing member 14. For example, the additional port 68 can provide access for one or more tools for accessing an internal speaker 28 (FIG. 1B), which can be configured to provide an audible indication, such as for when the device 2 is fully charged and/or in the treatment ready status, by way of non-limiting examples. It should be appreciated that the additional port 68 can have other uses.

With continuing reference to FIG. 4C, the insertion portion 56 of the battery housing 52 includes the one or more battery receptacles 58 that are configured to hold, interchangeably, at least two types of batteries 60. As used herein, the term “interchangeably” means “at different times” or “in different instances” without requiring any reconfiguration made to the supporting component(s). For example, with reference to a battery receptacle 58, when it is said that the battery receptacle 58 is configured to hold, interchangeable, at least two types of batteries 60, it is meant that the battery receptacle 58 is configured to hold a first type of battery (such as an Alkaline battery) in one instance and is also configured to hold a second type of battery (such as a nickel-metal-hydride (NiMH) battery) in another instance without requiring any reconfiguration made to the battery receptacle 58 or supporting components of the battery housing 52. Such battery interchangeability provides the user with options for selecting between the different battery types, which can be a significant advantage, particularly in areas where battery availability is limited. Additionally, as used herein, the phrases “type of battery” and “battery types” refer to the type of battery cell chemistry, i.e., Alkaline, nickel-metal-hydride (NiNM), lithium-ion (Li-Ion), nickel-zinc (NiZn), and nickel-cadmium (NiCd), and the like.

The battery receptacles 58 are configured to hold batteries of a particular size and a particular battery type. In the illustrated embodiment, the battery housing 52 has two (2) battery receptacles 58, each configured to hold a AA-sized battery. In this manner, the battery receptacles 58 are each configured to hold, interchangeably, a AA-sized alkaline battery 60 (e.g., 1.5 Volt) and a AA-sized nickel-metal-hydride (NiNM) battery 60 (e.g., 1.25 Volt). In other embodiments, the one or more battery receptacles 58 can each be configured to also hold, interchangeably, one or more additional battery types, such as AA-sized lithium (e.g., lithium-ion (Li-ion)), Zinc-carbon, nickel-zinc (NiZn), and nickel-cadmium (NiCd), by way of non-limiting examples. It should be appreciated that, in other embodiments, the battery receptacles 58 can be configured to hold batteries of various types and/or various sizes. For example, in other embodiments, the one or more battery receptacles 58 can each be configured to hold other battery sizes, such as AAAA-size, AAA-size, B-size, C-size, D-size, 9-Volt, or various button-cell sizes, by way of non-limiting examples. It should be appreciated that, in additional embodiments, the battery receptacle(s) 58 can also include size-adjustable (e.g., compliant) mechanisms for interchangeably holding batteries having different sizes, such as AAAA-size, AAA-size, AA-size, B-size, C-size, D-size, 9-Volt, and different battery types, such as alkaline, NiMH, Zinc-carbon, lithium (e.g., Li-ion), lithium-iron-phosphate (LiFePO₄ or “LFP”), lithium-polymer (e.g., LiPo), nickel-zinc (NiZn), nickel-cadmium (NiCd), and lead-acid (e.g., sealed lead-acid (SLA) and valve-regulated lead-acid (VRLA)), by way of non-limiting examples. As used herein with reference to a battery or battery type, the term “preselected” means that such battery or battery type is usable in the battery receptacles 58. Thus, when the following description refers to a “preselected” battery or batteries, such battery or batteries are usable in the battery receptacles 58.

The control unit 90, shown in FIG. 4A, controls the charge circuit 80 to deliver electric charge from the battery unit 50 to the energy storage unit 70. In particular, the control unit 90 is powered by the batteries 60 and is configured to manipulate the charge circuit 80 to direct electric charge from the batteries 60 to energy storage unit 70 in a manner providing enhanced charging of the energy storage unit 70. The energy storage unit 70 includes at least one energy storage device 72, such as a supercapacitor, a battery, a capacitor, a plurality of any such storage device, and/or various combinations of the foregoing, by way of non-limiting examples. The at least one energy storage device 72 can have a total capacitance (or total equivalent capacitance) in a range of about 0.01 Farad to about 250 Farads, and more particularly in a range of about 0.15 Farads to about 7.5 Farads, and more particularly in a range of about 1.20 Farads to about 1.30 Farads.

The at least one energy storage device 72 is configured to discharge an output signal having sufficient energy to electroporate tissue in vivo. In particular, the at least one energy storage device 72 is configured to discharge a plurality of energy pulses with sufficient energy to be converted by downstream component(s) into electroporation pulses. These energy pulses can be delivered in various pulse groups (i.e., sequences) that are configured to provide respective electroporation treatments. As used herein, the terms “pulse group” and “pulse sequence” refer to an energy pulse group having sufficient energy to electroporate tissue in vivo. It should be appreciated that each such pulse group or pulse sequence is configured to provide a respective electroporation treatment to a patient. It should also be appreciated that each such pulse group or pulse sequence can include a range of pulse quantities from one pulse (a single pulse) to more than one pulse, including two (2), three (3), four (4), five (5), six (6), seven (7), eight (8), nine (9), ten (10), and more than ten pulses. It should further be appreciated that each such pulse group or pulse sequence can include one or more additional pulses of energy that lack sufficient energy to electroporate tissue but that serve other purposes for the electroporation treatment, such as for measuring impedance or other parameters for providing active feedback information to the control unit 90, by way of a non-limiting example.

In this regard, the at least one energy storage device 72 is configured to discharge various output signals having energy characteristics useful for providing electroporation treatments. For example, the at least one energy storage device 72 can be configured to discharge one or more energy pulses each having an energy in a range from about 0.004 Joules to about 4.5 Joules. The at least one energy storage device 72 can also be configured to discharge pulse sequences (for electroporation treatments) in which each pulse sequence has a total energy in a range of about 0.015 Joules to about 15.0 Joules. It should be appreciated that the energy magnitude of the output signals can be adjusted as needed to tailor the pulse sequences for electroporating various types of tissues, including skin, adipose, muscle, and mucosal tissue.

In the illustrated embodiment, the energy storage unit 70 comprises a plurality of supercapacitors 72, particularly a pair of supercapacitors 72 connected in series to increase the voltage of their stored charge. In the illustrated embodiment, the supercapacitors 72 each have a capacitance of about 1.25 Farads and a maximum voltage of about 6.0 Volts, so that when connected in series the supercapacitors 72 have a total capacitance of about 1.25 Farads and a total voltage of about 12.0 Volts. One advantage of using one or more supercapacitors 72 as the energy storage unit 70 is that supercapacitors can discharge virtually all of their stored energy rapidly. Thus, the supercapacitors 72 of the illustrated embodiment can rapidly discharge an output signal having a total energy of about 90 Joules, various proportions of which (and up to virtually all of which) can be converted into electroporation pulses. In other embodiments, the energy storage unit 70 can be configured to discharge an output energy signal having a total energy in a range of about 7 Joules to about 150 Joules. Thus, in embodiments of the present disclosure, the energy storage unit 70 can be configured to discharge an output energy signal having a total energy of at least about 7 Joules, at least about 10 Joules, at least about 20 Joules, at least about 30 Joules, at least about 40 Joules, at least about 50 Joules, at least about 60 Joules, at least about 70 Joules, at least about 80 Joules, at least about 90 Joules, at least about 100 Joules, at least about 110 Joules, at least about 120 Joules, at least about 130 Joules, at least about 140 Joules, at least about 150 Joules, and yet greater values. It should be appreciated that various other supercapacitor configurations and values (e.g., capacitance and voltage) are within the scope of the present disclosure. It should also be appreciated that when the energy storage unit 70 includes one or more supercapacitors 72, the one or more supercapacitors 72 can be referred to as a “supercapacitor unit” 72.

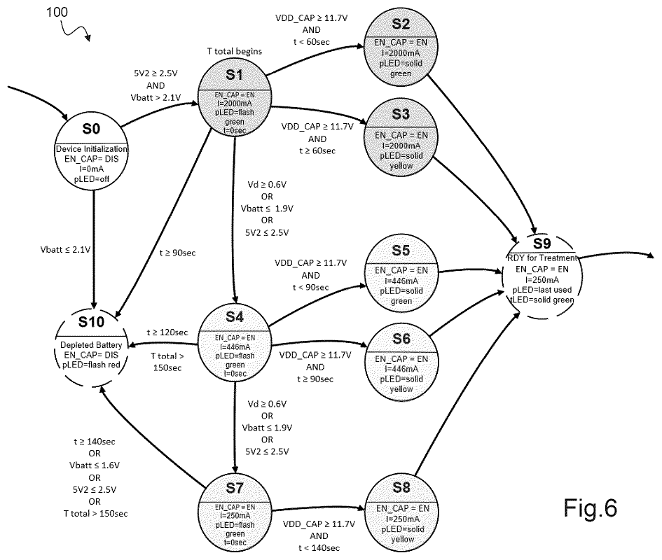

The control unit 90 executes machine-readable instructions, which include an algorithm 100 (FIG. 6) that is adapted to extract substantially the maximum energy (e.g., the highest charge current) at the highest efficiency from the batteries 60 (regardless of the type of preselected batteries 60 inserted in the battery housing 52) to charge the supercapacitors 72 without negatively impacting operation of the control unit 90, which is also powered by the batteries 60. This configuration is particularly advantageous for numerous reasons. One reason is that the supercapacitors 72 can store energy at a low voltage and produce a high, virtually instantaneous current, which is useful for generating electroporation pulses. Another reason is that the supercapacitors 72 have a significantly higher energy storage capacity (e.g., multiple Farads) compared to regular capacitors, which store micro-Farads of energy. The lower voltage of the supercapacitors 72 compared to regular capacitors can be partially mitigated by connecting the supercapacitors 72 in series. An additional advantage is that storing energy at low voltage with the supercapacitors 72 conserves overall space within the device 2, which allows the device 2 to have a smaller size compared to state of the art electroporation devices, which allows the device 2 to be mass produced at industrial scales (i.e., quantities), which is particularly advantageous for mass vaccination type settings. A yet further advantage of the device 2 according to the illustrated embodiments is that the devices 2 can be stored long-term in a stockpile type of setting and yet be usable with the preselected, widely available, off-the-shelf battery types described above, which can be easily purchased by an end-use at most locations across the globe. Yet another advantage is that the algorithm 100, by increasing the efficiency at which energy is drawn from the batteries, thereby increases the life of the batteries, and also provides added safety benefits by protecting the batteries from overstress, overheating, inefficient or insufficient output, catching fire and/or other potential damage. Yet further advantages of using replaceable batteries is that a user can charge batteries externally and have them ready for replacing used batteries in the device 2 as needed, such as in a high-use scenario such as mass vaccination settings. These advantages effectively combine to provide a small, self-contained, storable, widely usable handheld electroporation device 2. It should be appreciated that, to ensure that batteries don't leak, the devices 2 herein can be stored without batteries.

It should also be appreciated that the power-related advantages described above provide the device 2 with a smaller size and lighter weight relative to prior art devices. These size and weight savings allow the device 2 to have enhanced ergonomic features, which in combination with the size and weight savings provide further advantages regarding usability, particularly in high-use settings, such as mass vaccination settings. These usability advantages include reducing operator fatigue. In particular, the application angle A1 and pistol-grip design for the delivery trigger 20 allows the user's wrist to be held substantially perpendicular to the patient's arm during treatment, which has been observed to reduce the strain on the user's wrist. The device 2 of the illustrated embodiment also enables better visibility of the treatment site (e.g., injection bleb), because the bulk of the device 2 (e.g., the first housing member 14) is substantially at a right angle to the treatment site and the electrode array 34 tapers distally. These features provide better visibility of the drug injection site, allowing for better co-location of the electrode(s) 10 to be centered into the injection bleb. The device 2 can be held by the user in both a sitting and standing position, again reducing user fatigue, such as at the user's wrist. These features have been observed to make it easier for users to maintain the electrode(s) 10 at a proper insertion angle and insertion depth in patient tissue. It should be appreciated that during treatment, the electroporation pulse(s) typically cause the patient's muscle to contract with each pulse, causing the patient's arm to flex. For optimal treatment, it is therefore critical for the user to maintain the electrode(s) 10 at the proper insertion depth in the target tissue. For example, if the electrode(s) back out a small amount, the impedance caused by the tissue reduces, thereby increasing the electric current at the electrode 10, which can cause poor transfection and potential burning at the electrode insertion point. By being lightweight and favorably sized, and by having an easy to maintain insertion depth and application angle A1, the foregoing challenges are more readily avoidable.

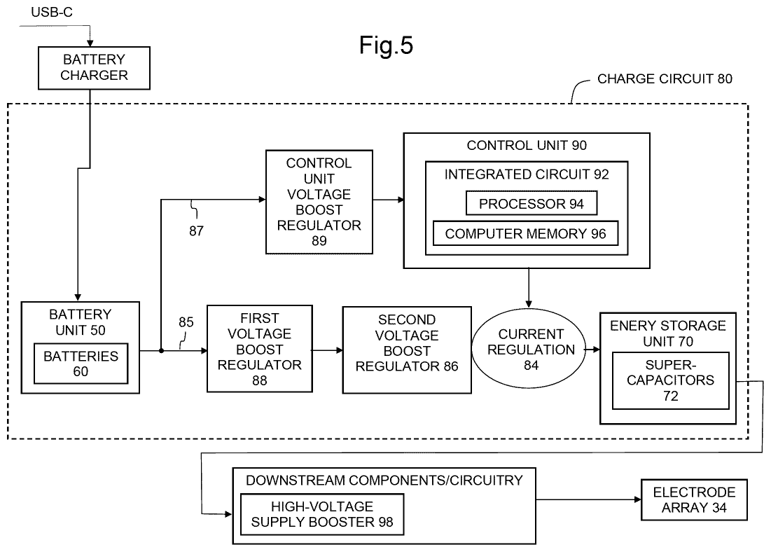

Referring now to FIG. 5, the charge circuit 80 of the illustrated embodiments includes the battery unit 50, the supercapacitors 72, and the control unit 90. The control unit 90 performs current regulation 84 operations to control the electrical current drawn from the batteries 60 (referred to herein as the “charge current”) for delivery to the supercapacitors 72. The charge circuit 80 can also include at least one voltage boost regulator 86 located between the battery unit 50 and the supercapacitors 72 for increasing (e.g., “boosting”) the voltage of the battery signal delivered to the supercapacitors 72. The use of such voltage boost regulators(s) 86 can increase the voltage from the battery voltage to a boosted voltage, which is preferably substantially equivalent to a voltage of the supercapacitors 72 when fully charged, which can be referred to as the “full supercapacitor voltage”. By way of non-limiting examples, the boosted voltage can be in a range from about 5.0 Volts to about 20.0 Volts, and more particularly in a range from about 10.0 Volts to about 14.0 Volts, and more particularly in a range from 11.5 Volts to about 12.5 Volts. In one particular non-limiting example, the target boosted voltage is from about 11.7 Volts to about 12 Volts, which can be tailored to the voltage capacity of the plurality of supercapacitors 72.

In additional embodiments, the charge circuit 80 can include at least one additional voltage boost regulator 88. In such embodiments, the additional voltage boost regulator 88 can be located between the battery unit 50 and the voltage boost regulator 86. In such embodiments, the additional voltage boost regulator 88 can be referred to as a “first voltage boost regulator” 88, and the other can be referred to as a “second voltage boost regulator” 86. Additionally, the first voltage boost regulator 88 can be said to boost the voltage of the charge current from the battery voltage to a first boosted voltage, and the second voltage boost regulator 86 can be said to boost the voltage of the charge current from the first boosted voltage to a second boosted voltage (i.e., the full supercapacitor voltage). It should be appreciated that voltage boost regulators tend to operate more efficiently at higher input voltages; therefore the use of the first and second voltage boost regulators 88, 86 can increase the efficiency of the second voltage boost regulator 86, which boosts the voltage to the full supercapacitor voltage. By way of non-limiting examples, the second boosted voltage can be in the ranges described above with reference to the boosted voltage, and the first boosted voltage can be in a range from about 2.0 Volts to about 10.0 Volts, and more particularly in a range from about 3.5 Volts to about 7.0 Volts, and more particularly in a range from 4.8 Volts to about 5.6 Volts. In one particular non-limiting example, the first boosted voltage is about 5.2 Volts, and the second boosted voltage is about 11.84 Volts.

The first and second voltage boost regulators 88, 86 are located along a first, primary branch 85 of the charge circuit 80 extending from the battery unit 50 to the energy storage unit 70. The control unit 90 is disposed along a second, control branch 87 of the charge circuit 80 that extends from the battery unit 50 to the control unit 90 and is parallel with the first branch 85. It should be appreciated that the current regulation 84 can be performed by the second voltage boost regulator 86, as controlled by the control unit 90. In the illustrated embodiment, the control branch 87 includes a control unit voltage boost regulator 89 interposed between the battery unit 50 and the control unit 90. The control unit voltage boost regulator 89 is configured to boost the voltage along the control branch 87 from the battery voltage to boosted control voltage, which is at least equivalent to a minimum operational voltage of the control unit 90. The boosted control voltage can be in a range of about 1.7 Volts to about 7.0 Volts, and more particularly in a range of about 2.0 Volts to about 5.0 Volts, and more particularly in a range of about 3.0 Volts to about 3.5 Volts. In one particular non-limiting example, the boosted control voltage is about 3.3 Volts.

The control unit 90 can include an integrated circuit 92, which can include a processor 94, such as a microprocessor 94, for executing the machine-readable instructions (e.g., the algorithm) and to perform current regulation 84 operations for controlling the charge current of the battery signal delivered to the supercapacitors 72. The control unit 90 can also include computer memory 96, such as for storing the machine-readable instructions. The computer memory 96 is in electric communication with the processor 94 and can be incorporated into the integrated circuit 92. The integrated circuit 92 can be a 32-bit ARM core type of microcontroller, by way of a non-limiting example.

The supercapacitor unit 72 outputs the charged electrical energy stored therein (e.g., at the full supercapacitor voltage) in an output signal transmitted to downstream components that tailor the output signal into an electroporation signal. One such downstream component is a high-voltage supply booster 98, which boosts the voltage of the output signal (e.g., the full supercapacitor voltage) to an electroporation voltage, which can be in a range from about 5 Volts to about 1000 Volts (1 kV), and more particularly in a range of about 100 Volts to about 400 Volts, and more particularly in a range of about 175 Volts to about 250 Volts. In one particular non-limiting example, the electroporation voltage is in a range of about 190 Volts to about 210 Volts. Additional downstream components include safety circuitry, one or more fuses, an additional electric current (amperage) regulator, active feedback circuitry, and a pulse switch matrix for controlling the pulse firing pattern of the electrodes 10, which firing patterns are described in more detail below. The one or more electroporation pulses delivered to the electrodes 10 can have an electric current magnitude in a range from about 0.01 Amp to about 2.0 Amps, and more particularly in a range of about 0.05 Amp to about 0.5 Amp, and more particularly in a range of about 0.15 Amp to about 0.25 Amp. The one or more electroporation pulses can each have a pulse duration in a range of about 100 microseconds (μs) to about 500 milliseconds (ms), and more particularly in a range of about 1.0 milliseconds (ms) to about 100 milliseconds (ms), and more particularly in a range of about 40 milliseconds (ms) to about 60 milliseconds (ms). The quantity of electroporation pulses can be in a range of 1 pulse to about 10 pulses, and more particularly in a range of about 3 pulses to about 5 pulses. For multi-pulse deliveries, each electroporation pulse can be separated in time from adjacent pulses by a pulse delay in a range of about 1 millisecond to about 5 seconds.

It should be appreciated that, in embodiments with multiple electrodes, the one or more electroporation pulses can be delivered according to various pulse firing patterns, in which various pulses in the pulse sequence are delivered to various, predetermined electrodes 10. In one particular non-limiting example, the device 2 has three (3) electrodes arranged spatially in a triangular pattern. In this particular example, the electroporation pulses are delivered in a sequence of pulses, such that during each pulse in the sequence, one of the electrodes 10 is positive or “active” (i.e., delivers the pulse to the tissue), one of the electrodes 10 is negative (i.e., a return electrode), and the other electrode 10 is floating or neutral (e.g., can be used for measuring impedance for providing feedback). In such examples, the pulse firing pattern can employ different electrodes 10 as the positive, negative, and neutral electrodes 10 for successive pulse(s) in the pulse sequence. In other example embodiments, a pulse in a pulse sequence can be delivered from two positive electrodes to a single negative electrode. In yet another example embodiments, a pulse in a pulse sequence can be delivered from one positive electrode to two negative electrodes. In further example embodiments, the device 2 can have four or more electrodes 10. In such embodiments, any pulse in the pulse sequence can utilize one or more positive electrodes 10, one or more negative electrodes 10, and one or more neutral electrodes 10. It should be appreciated that the output signal discharged by the supercapacitor unit 72 can be tailored as needed by the downstream components to provide any desirable pulse firing pattern through the electrodes 10.

In one particular non-limiting example of the illustrated embodiments, the device 2 has three (3) electrodes 10 that deliver a series of four (4) electroporation pulses each having a voltage of in a range of about 180 Volts to about 220 Volts, an electric current magnitude in a range of about 0.18 Amp to about 0.22 Amp, and a pulse duration in a range of about 40 milliseconds (ms) to about 60 milliseconds (ms), with an inter-pulse delay between the pulses in a range of about 200 milliseconds (ms) to about 3.5 seconds. It should be appreciated that various other pulse parameters (e.g., voltage, current magnitude, pulse duration, and inter-pulse delay) are within the scope of the present disclosure. It should also be appreciated that the device 2 can be configured to deliver one or more additional, non-electroporative pulses before and/or between the electroporation pulse(s). One such example of non-electroporative pulses involves the delivery of one or more impedance pulses (i.e., for measuring impedance in the target tissue) to provide feedback data to the control unit 90, which interprets the feedback data to adjust the electroporation pulse(s) in real-time, thereby providing the device 2 with a feedback mechanism. Such feedback data can be used to maintain the electroporation pulse(s) delivered to the tissue at a constant current magnitude, as more fully described in International Publication No. WO 2008/048632, published Apr. 24, 2008, entitled “ELECTROPORATION DEVICES AND METHODS OF USING SAME FOR ELECTROPORATION OF CELLS IN MAMMALS” (“the '632 Reference”), and/or to determine the type of tissue (e.g., skin, fat, or muscle) in contact with the electrode tips, as more fully described in U.S. Pat. No. 10,610,684, issued Apr. 7, 2020, entitled “VARIABLE CURRENT DENSITY SINGLE NEEDLE ELECTROPORATION SYSTEM AND METHOD” (“the '684 Reference”), the entire disclosure of each of which are hereby incorporated by reference herein.

Referring now to FIG. 6, an example of the algorithm 100 for charging the energy storage unit 70 from the batteries 60 is shown. The algorithm 100 is specifically configured to instruct the control unit 90 to operate the charge circuit 80 to optimize the charging of the energy storage unit 70, such as the supercapacitors 72 (or, in other embodiments, a bank of capacitors, batteries, or combinations of the foregoing). The algorithm 100 instructs the control unit 90 to effectively extract the maximum energy (in the illustrated example, the highest charge current), efficiently, out of the batteries 60 without dropping the charge voltage below the minimum operational voltage of the control unit 90 (e.g., about 3.3 Volts for the non-limiting example described above). The algorithm 100 also instructs the control unit 90 to communicate various indication signals or outputs to the user indicating various charge statuses of the device 2. These indication outputs include optical outputs via the power LED 25 and the treatment LED 26 and can optionally include one or more audible outputs via the speaker 28 (see FIGS. 1A-1).

It should be appreciated that the example algorithm 100 shown in FIG. 6 is tailored according to one non-limiting example implementation of the present disclosure. In particular, this non-limiting example implementation involves charging an energy storage unit 70 having a pair of supercapacitors 72 connected in series from a pair of AA-sized batteries 60, which are interchangeably a pair of NiMH or a pair of alkaline batteries. Furthermore, the algorithm 100 is specifically tailored to charge the supercapacitors 72 using these multiple types of batteries 60 (NiMH and Alkaline) and to adjust the charge current in response to the charge level of the batteries 60. It should be appreciated that although the following description of the algorithm 100 refers specifically to the illustrated embodiment of the algorithm 100 based on the example non-limiting implementation, various features of the algorithm 100 can be adjusted for the example implementation and/or for other implementations while remaining within the scope of the present disclosure. For example, although the illustrated embodiment of the algorithm 100 is tailored for use with AA-sized NiMH and Alkaline batteries interchangeably, the algorithm 100 can be adjusted for use with other power output requirements and other battery sizes and types, such as lithium-ion (Li-Ion), nickel-zinc (NiZn) and nickel-cadmium (NiCd) batteries, by way of non-limiting examples Non-limiting examples of possible variations to the algorithm 100 will be discussed in more detail below.

The algorithm 100 is configured to transition the charge current between various charge states of a plurality of potential charge states responsive to (i.e., as determined by) condition statement outcomes that employ measured input parameters of the charge current as conditions of the condition statements. The illustrated algorithm 100 employs charge states S0, S1, S2, S3, S4, S5, S6, S7, S8, S9, and S10. Charge state S0 can be referred to as an “initialization” or “initial” state S0, which automatically occurs when the device 2 is turned ON via the power button 18. After initialization S0 is complete, the algorithm 100 transitions the charge current selectively among various sequences of charge states S1-S8 to either charge the supercapacitors 72 to charge state S9 (in which the supercapacitors 72 are at the treatment ready status, meaning ready to discharge the output signal for generating the electroporation pulse(s)) or to arrive at charge state S10, in which the control unit 90 determines that the batteries 60 lack sufficient charge to charge the supercapacitors 72 without dropping below the minimum operational voltage of the control unit 90. At charge state S10, the control unit 90 ceases charging the supercapacitors 72 and indicates to the user that the batteries 60 collectively are depleted. Charge state S10 can thus be referred to as a “Depleted Battery” state and/or an “abort” state. Charge state S10 provides an important safety feature because, among other things, if the charge current causes the voltage to drop below the minimum operational voltage of the control unit 90, the control unit 90 cannot reliably monitor the input parameters during supercapacitor charging, which can lead to various components overheating and potentially damaging the device 2 if left uninterrupted.

The algorithm 100 of the illustrated embodiment employs two general modes of active charging: (1) primary charging (states S1, S4, and S7), in which the batteries 60 charge the supercapacitors 72 toward a full charge level (i.e., “full charge”); and (2) top-off charging (state S9), which occurs after the supercapacitors 72 reach a minimum threshold charge level that is substantially near their full charge level. For purposes of this disclosure, when the supercapacitors 72 are at or above the minimum threshold charge level (as measured by voltage), the supercapacitors 72 can be characterized as fully charged. During top-off charging (state S9), the charge current is employed to maintain the supercapacitors 72 substantially at full charge (i.e., constant voltage). In the illustrated embodiment, once the charge current enters top-off charging (state S9), the device 2 is treatment ready. Thus, charge state S9 can be referred to synonymously as the “top-off” and “treatment ready” charge state S9. The algorithm 100 of the illustrated embodiment also includes indication charge states S2, S3, S5, S6, S8, which indicate to the user the transition from primary charging S1, S4, S7 to top-off charging S9. Moreover, at the indication charge states S2, S3, S5, S6, S8, the algorithm 100 instructs the control unit 90 to indicate to the user (via the power LED 25) whether the batteries 60 have generally a normal charge level or a low charge level upon charging the supercapacitors 72 to full charge. In some embodiments of the algorithm 100, the indication charge states S2, S3, S5, S6, S8 can be said to occur momentarily between primary charging S1, S4, S7 and top-off charging S9.

In the illustrated embodiment, the algorithm 100 selects between three (3) potential primary charge states S1, S4, S7 for charging the supercapacitors 72, each primary charge state S1, S4, S7 utilizing a different maximum charge current magnitude (e.g., high, medium, and low current), which are tailored to accommodate preselected battery types and associated battery charge levels. The algorithm 100 is configured to transition between the primary charge states S1, S4, S7 from high-current (S1) to medium-current (S4) to low-current (S7) primary charging as necessary to charge the supercapacitors 72 while maintaining at least the minimum operational voltage of the control unit 90. It should be appreciated that the following example current magnitudes employed for the primary charge states S1, S4, and S7 are based on the non-limiting example implementation described above; in other embodiments of the algorithm 100, different maximum current magnitudes can be employed.

Primary charge state S1 employs a charge current of about 2000 mAmps (2 Amps), which is the highest charge current of the primary charge states; S1 can thus also be characterized as the “high charge” primary charge state S1. A charge current of 2000 mAmps is particularly beneficial for use with power sources that can handle high current discharges (e.g., charging currents), such as NiMH batteries.

Primary charge state S4 employs a charge current of about 446 mAmps (0.446 Amp) and can also be referred to as the “medium charge” primary charge state S4. A charge current of about 446 mAmps (0.446 Amp) can be employed beneficially for use with both NiMH and Alkaline batteries 60. For example, a charge current of about 446 mAmps can provide a step-down charge current (from S1) for NiMH batteries 60. Additionally, Alkaline batteries 60 having a full or high battery charge level can discharge 446 mAmps while maintaining at least the minimum operational voltage of the control unit 90.

Primary charge state S7 employs a charge current of about 250 mAmps (0.250 Amp) and can also be referred to as the “low charge” primary charge state S7. A charge current of 250 mAmps is particularly beneficial for use extracting the remaining charge available within NiMH batteries 60 and providing a step-down charge current (from S4) for Alkaline batteries 60 while maintaining at least the minimum operational voltage of the control unit 90.

The algorithm 100 employs the following measured input parameters to determine the transition pathway between the charge states S0-S10:
(A) battery voltage (“Vbatt”), i.e., the battery voltage as measured during the respective charge state. The battery voltage (Vbatt) is measured at a first location, which can be at the battery output terminals 62 or between the output terminals 62 and the first voltage boost regulator 88.
(B) battery delta voltage (“Vd”), i.e., the change/drop in battery voltage when a load (i.e., the supercapacitors 72) is applied to the batteries 60. The change/drop in battery voltage is calculated by measuring the ongoing voltage of the batteries as they charge the supercapacitors 72 and comparing the ongoing values to a baseline, no-load value for the battery voltage (Vbatt). The battery delta voltage (Vd) can be measured at the first location.
(C) first boosted voltage (“5V2”), i.e., the boosted voltage produced by the first voltage boost regulator 88, measured at a second location that is at the first voltage boost regulator 88 or between the first and second voltage boost regulators 88, 86.
(D) second boosted voltage (“VDD_CAP”), i.e., the boosted voltage produced by the second voltage boost regulator 86, measured at a third location that is at the second voltage boost regulator 86, at the supercapacitors 72, or at an intermediate location therebetween (in the illustrated embodiments, the second boosted voltage is the voltage entering the supercapacitors 72);
(E) state charging duration (“t”), i.e., the charging duration in the specific primary charge state S1, S4, S7.
(F) total charging duration (“T total”), i.e., the total primary charging duration beginning at the commencement of the High-Current Primary Charge state S1.
   It should be appreciated that, by measuring these input parameters, the control unit 90 (as instructed by the algorithm 100) effectively ascertains the type of batteries 60 employed in the battery housing 50 and their collective battery charge level.

Further description of the algorithm 100 will now be described with continuing reference to FIG. 6. When the device 2 is turned ON, the control unit 90 executes the algorithm 100, which begins at the initialization charge state S0, in which no charge current is drawn from the batteries 60 and in which the power LED 25 is in the OFF state. At Initialization S0, the control unit 90 begins measuring the battery voltage (Vbatt) at the first location and also measures the first boosted voltage (5V2) at the second location to determine whether the measured values equal or exceed respective, predetermined threshold values, which act as preconditions for triggering a transition to either S1 or S10. In particular, the control unit 90 samples the battery voltage (Vbatt) and first boosted voltage (5V2) at sample intervals of 100 milliseconds (ms). If the control unit 90 determines that the battery voltage (Vbatt) does not exceed (i.e., is less than or equal to) a threshold value of 2.1 Volts, the charge state transitions from S0 to the Depleted Battery state S10. At the Depleted Battery state S10, the control unit 90 causes the power LED 25 to flash red, indicating to the user that the batteries 60 lack sufficient battery charge to charge the superconductors 72. This instructs the user to either charge the batteries 60 via USB-C port 67 or to replace the batteries with different NiMH or Alkaline batteries 60 and begin again. If, however, during Initialization S0, the control unit 90 determines that the battery voltage (Vbatt) exceeds the threshold value of 2.1 Volts and that an average of the first boosted voltage (5V2) measurements equals or exceeds a threshold value of 2.5 Volts, the charge current transitions to S1 (High-Current Primary Charging).

At the commencement of High-Current Primary Charging S1, the charge circuit 80 draws current from the batteries and delivers a charge current of 2000 mAmps to the supercapacitors 72. Additionally, the control unit 90 begins measuring the state charging duration t and the total charging duration T. The control unit 90 also causes the power LED 25 to flash green, indicating to the user that the device 2 is in one of the primary charge states S1, S4, S7. The control unit 90 continues sampling the battery voltage (Vbatt) and the first boosted voltage (5V2) at intervals of 100 milliseconds (ms). The control unit 90 also begins measuring the battery delta voltage (Vd) and the second boosted voltage (VDD_CAP) (the latter measured at the third location) at the sample intervals (100 ms). Under the foregoing conditions, if the control unit 90 determines that the second boosted voltage (VDD_CAP) equals or exceeds (i.e., reaches or attains) a minimum threshold voltage of 11.7 Volts (i.e., substantially equivalent to the full supercapacitor voltage) before the state charging duration t equals a first threshold state charging duration of 60 seconds, the charge current transitions to indication charge state S2, so long as none of the following three trigger conditions occurs before the first threshold charging duration (t≤60 seconds): (1) the battery delta voltage (Vd) equals or exceeds a threshold delta voltage of 0.6 Volt; or (2) the battery voltage (Vbatt) equals or falls below a threshold voltage of 1.9 Volts; (3) or the first boosted voltage (5V2) equals or falls below a threshold voltage of 2.5 Volts, as discussed further below.

At Indication Charge State S2, the algorithm 100 instructs the control unit 90 to cause the power LED 25 to radiate solid green, indicating to the user that the supercapacitors 72 are fully charged and that the batteries 60 remain at least at a medium charge level. The power LED 25 continues to radiate solid green throughout the top-off charge state S9. If, however, the control unit 90 determines that the second boosted voltage (VDD_CAP) reaches the threshold voltage of 11.7 Volts only at or after passage of the first threshold state charging duration (t=60 seconds) but before the state charging duration t equals a second threshold state charging duration of 90 seconds (t=90 s), and so long as none of the aforementioned three trigger conditions occurs, the algorithm 100 instructs the control unit 90 to transition the charge current to indication charge state S3. At S3, the power LED 25 radiates solid yellow, indicating to the user that the supercapacitors 72 are fully charged but that the batteries 60 are at a low battery charge level following supercapacitor charging. Thereafter, the power LED 25 continues to radiate solid yellow throughout the top-off charge state S9.

Referring again to the high-current primary charge state S1, if the state charging duration t equals or exceeds the second threshold state charging duration of 90 seconds (t=90 s) before the second boosted voltage (VDD_CAP) can equal the threshold voltage of 11.7 Volts (i.e., before the supercapacitors 72 reach full charge) and before any of the three trigger conditions occurs, the control unit 90 transitions the charge current to the Depleted Battery state S10. If, however, any of the three trigger conditions occurs before the second boosted voltage (VDD_CAP) reaches the threshold voltage of 11.7 Volts and before expiration of the third threshold state charging duration (t=90 s), the control unit 90 transitions the charge current to the medium-current primary charge state S4.

At the commencement of the medium current primary charge state S4, the charge current is reduced to about 446 mAmps and the control unit 90 begins measuring a respective state charging duration t (i.e., the control unit 90 begins measuring a new state charging duration t starting at t=0). The control unit 90 continues measuring the total charging duration T that commenced at the beginning of state S1. The power LED 25 continues to flash green, indicating that the device 2 remains in one of the primary charge states S1, S4, S7. The control unit 90 can restart sampling of one or more of the input parameters. For example, the control unit 90 can restart sampling the battery delta voltage (Vd) and the first boosted voltage (5V2) at intervals of 100 milliseconds (ms) while sampling continues for the second boosted voltage (VDD_CAP) and the battery voltage (Vbatt). Under the foregoing conditions, if the control unit 90 determines that the second boosted voltage (VDD_CAP) reaches the threshold voltage of 11.7 Volts (i.e., the supercapacitors 72 reach full charge) before the state charging duration t equals a first threshold state charging duration of 90 seconds (t=90 s) and before the total charging duration T exceeds a total charging duration threshold of 150 seconds (T>150 s), and so long as none of the aforementioned three trigger conditions occurs beforehand, the charge current transitions to indication charge state S5.

At charge state S5, the power LED 25 radiates solid green, indicating to the user that the supercapacitors 72 are fully charged and that the batteries 60 remain at least at a medium charge level. Thereafter, the power LED 25 continues to radiate solid green throughout the top-off charge state S9. If, however, the control unit 90 determines that the second boosted voltage (VDD_CAP) reaches the threshold voltage of 11.7 Volts (i.e., the supercapacitors 72 reach full charge) at or after passage of the first threshold state charging duration (t=90 s) but before the state charging duration t equals a second threshold state charging duration of 120 seconds (t=120 s) and before the total charging duration T exceeds the total charging duration threshold of 150 seconds (T>150 s), and so long as none of the aforementioned three trigger conditions occurs, the algorithm 100 instructs the control unit 90 to transition the charge current to indication charge state S6. At S6, the power LED 25 radiates solid yellow, indicating to the user that the supercapacitors 72 are fully charged but that the batteries 60 are at a low battery charge level following supercapacitor charging. Thereafter, the power LED 25 continues to radiate solid yellow throughout the top-off charge state S9.

Referring again to the medium-current primary charge state S4, if the second boosted voltage (VDD_CAP) does not reach the threshold voltage of 11.7 Volts (i.e., the supercapacitors 72 do not reach full charge) and none of the three trigger conditions occurs before the state charging duration t equals or exceeds the second threshold state charging duration of 120 seconds (t=120 s) or before the total charging duration T exceeds the total charging duration threshold of 150 seconds (T=150 s), the algorithm 100 instructs the control unit 90 to transition the charge current to the Depleted Battery state S10. If, however, any of the three trigger conditions occurs before the second boosted voltage (VDD_CAP) reaches the threshold voltage of 11.7 Volts (i.e., before the supercapacitors 72 reach full charge) and before expiration of the second threshold state charging duration (t=120 s) and before expiration of the total charging duration threshold of 150 seconds (T=150 s), the algorithm 100 instructs the control unit 90 to transition the charge current to the low-current primary charge state S7.

At the commencement of the low-current primary charge state S7, the charge current is reduced to about 250 mAmps and the control unit 90 begins measuring a respective state charging duration t (i.e., the control unit 90 begins measuring a new state charging duration t starting at t=0). The control unit 90 continues measuring the total charging duration T that commenced at the beginning of state S1. The power LED 25 continues to flash green, indicating that the device 2 remains in one of the primary charge states S1, S4, S7. The control unit 90 restarts sampling the battery delta voltage (Vd) and the first boosted voltage (5V2) at intervals of 100 milliseconds (ms) while sampling continues for the second boosted voltage (VDD_CAP) and the battery voltage (Vbatt). Under the foregoing conditions, if the control unit 90 determines that the second boosted voltage (VDD_CAP) reaches the threshold voltage of 11.7 Volts (i.e., the supercapacitors 72 reach full charge) before the state charging duration t reaches a threshold of 140 seconds (t=140 s) and before the total charging duration T exceeds a threshold of 150 seconds (T=150 s), the algorithm 100 instructs the control unit 90 to transition the charge current to indication charge state S8, so long as neither of the following two trigger conditions occurs beforehand: (1) the battery voltage (Vbatt) equals or falls below a threshold voltage of 1.6 Volts; or (2) the first boosted voltage (5V2) equals or falls below a threshold voltage of 2.5 Volts. At indication charge state S8, the power LED 25 radiates solid yellow, indicating to the user that the supercapacitors 72 are fully charged but that the batteries 60 are at a low battery charge level following supercapacitor charging. Thereafter, the power LED 25 continues to radiate solid yellow throughout the top-off charge state S9. If, however, either of the aforementioned two trigger conditions occur at the low-current primary charge state S7, the algorithm 100 instructs the control unit 90 to transition the charge current to the Depleted Battery state S10. For the low-current primary charge state S7, the battery voltage (Vbatt) threshold of 1.6 Volts of the illustrated embodiment was selected because it provides a margin of safety over 0.5 Volts, which represents the minimum battery voltage required so that all electrical components of the device 2 operate as intended. A battery voltage (Vbatt) of 1.6 Volts while under load (i.e., while charging the supercapacitors 72) provides enough margin to cause the power LED 25 to flash red, thereby indicating that the batteries 60 are depleted and need to be recharged or replaced. It should be appreciated that, by transitioning the charge current to lower-current charge states if the various thresholds are triggered, the algorithm 100 also effectively provides safety protections that prevent, or at least significantly reduce the possibility of, the batteries from overstress and potential overheating, which could otherwise have negative consequences, particularly if overheated batteries produce inefficient or insufficient output, or worse, became damaged, corroded, catch-fire, and/or result in damage to the device 2.

At the top-off charge state S9, the algorithm 100 instructs the control unit 90 to transmit the charge current to the supercapacitors 72 at about 250 mAmps. In the illustrated embodiment, the top-off charge current of about 250 mAmps was selected for numerous reasons, including for simplicity, considering that 250 mAmps is also the charge current employed during primary charge state S7 in the illustrated example. The algorithm 100 can instruct the control unit 90 to employ the top-off charge current (about 250 mAmps) to maintain the supercapacitors 72 at a full charge level, which, for the illustrated example, is between the minimum threshold value of 11.7 Volts and an upper voltage setting of 11.84 Volts. It should be appreciated that, although the supercapacitors 72 of the illustrated embodiment have a maximum (fully charged) voltage of 12.0 Volts, the algorithm 100 imposes the upper voltage limit of 11.84 Volts during top-off charging S9 to elongate the service life of the supercapacitors 72. It should be appreciated that the top-off charge current magnitude can be adjusted as needed based on the particular implementation. It should also be appreciated that, for the illustrated example, after the supercapacitors 72 reach full charge, and in the absence of a load applied thereto, the supercapacitors 72 draw significantly less current than 250 mAmps to maintain full charge.

Also at the top-off charge state S9, the algorithm 100 instructs the control unit 90 to maintain the power LED 25 at the same indication from the respective indication charge state S2, S3, S5, S6, or S8 from which the charge signal transitioned. Thus, if the charge current transitioned to the top-off charge state S9 from S2 or S5, the power LED 25 will radiate solid green throughout top-off charging, whereas if the charge current transitioned to the top-off charge state S9 from S3, S6, or S8, the power LED 25 will radiate solid yellow throughout top-off charging. Additionally, at the top-off charge state S9, the algorithm 100 instructs the control unit 90 to cause the treatment LED 26 to radiate solid green, indicating that the device 2 is ready for treatment, meaning that the supercapacitors 72 are ready, upon the user activating the delivery trigger 20, to discharge the output signal to the downstream components which, in turn, convert the output signal into the one or more electroporation pulses. Subsequent to the supercapacitors 72 discharging the output signal, the algorithm 100 instructs the control unit 90 to terminate supercapacitor charging. By way of a non-limiting example, the control unit 90 terminates the supercapacitor charging after one or more impedance test pulses are delivered to the tissue yielding successful results and before the first electroporation pulse in the sequence is delivered to the tissue. Preferably, after the output signal is discharged, the algorithm 100 instructs the control unit 90 to turn OFF the device 2, so that the device 2 remains OFF until a user re-initiates the charge sequence by pressing the power button 18 to turn the device 2 ON. This acts as a single-use safety feature for the device 2.

It should be appreciated that the foregoing high, medium, and low charge currents (i.e., about 2000 mAmps, about 446 mAmps, and about 250 mAmps, respectively) were selected for the specific, non-limiting example of the algorithm 100 for the illustrated embodiment, and these current values were selected for specific purposes and considerations relating to user interface parameters (e.g., power LED and treatment LED) and relating to optimizing battery capacity. It should also be appreciated that the foregoing input parameters (e.g., battery voltage (Vbatt), battery delta voltage (Vd), first boosted voltage (5V2), second boosted voltage (VDD_CAP), state charging duration (t), and total charging duration (T)) and their respective threshold values were selected based on specific purposes and considerations relating to the illustrated embodiment. Non-limiting examples of such purposes and considerations will now be described.

S1, High-Current (2000 mAmps) Primary Charging:

Primary charge state S1 is intended for power sources that can handle high current discharges, such as NiMH batteries, and also alkaline batteries at a high battery charge level. The 2000 mAmps charge current was chosen as the highest current to use for the illustrated embodiment. This current level is intended for use with NiMH batteries. NiMH batteries can withstand discharge levels for 2000 mAmps supercapacitor charging for the majority (˜64%) of their battery capacity. If the battery voltage (Vbatt) during this high charge current drops too low or the battery delta voltage (Vd) rises too high, the act of transitioning to S4 and potentially to S7 allows the charge circuit 80 to utilize the remaining (˜36%) charge. The charge current of 2000 mAmps is also useful with Alkaline batteries having a high battery charge level, though even fully-charged Alkaline batteries tend to trigger transition to primary charge state S4 after about 4-6 uses.

It should be appreciated that, as the batteries are drained during S1 charging and the battery voltage inherently drops, the current drawn from the batteries 60 must increase to maintain the power required by the first voltage boost regulator 88 (5V2) for high-current charging S1. If this power becomes too much for the batteries to discharge without collapsing the first boost regulator output (5V2), then the supercapacitor charging ceases (i.e., the charge current transitions to S10). However, if the battery voltage (Vbatt), battery delta voltage (Vd), and first boosted voltage (5V2) remain within their thresholds, the high-current primary charging S1 will continue until the supercapacitors reach their full charge voltage level (at least 11.70 Volts) or until the state charge duration (t) exceeds its threshold. The inventors have determined through testing that, on average, it takes about 21 seconds to charge the supercapacitors at high-current charging S1 using NiMH batteries.

S4, Medium-Current (446 mAmps) Primary Charging:

Primary charge state S4 employs a lower charge current than S1 and is intended for use with both NiNM batteries and Alkaline batteries. The 446 mAmps charge level was chosen for S4 in the illustrated embodiment because it is an appropriate step-down current for NiNM batteries (e.g., to discharge a remaining 30% of their capacity before requiring a lower charge current) and also because Alkaline batteries can handle the required power output to keep the first voltage boost regulator (5V2) from collapsing. The inventors have determined through testing that, on average, it takes about 45 seconds (total) to charge the supercapacitors 72 when a transition from S1 to S4 is involved (as aggregated for both NiNM and Alkaline batteries), with individual charge times ranging from about 30 seconds to about 140 seconds when S4 is employed at any point in the charging process.

S7, Low-Current (250 mAmps) Primary Charging:

Primary charge state S7 is the lowest charge current that will be employed in the illustrated embodiment if any of the battery voltage (Vbatt), battery delta voltage (Vd), or first boosted voltage (5V2) fall outside their thresholds during S4. The 250 mAmps charge level was chosen for S7 in the illustrated embodiment because it further reduces the charge load so that NiNM batteries can utilize the last remainder of their charge, and also so that Alkaline batteries have a secondary primary charge level to operate in. For example, it has been observed through testing that applying a charge current of 250 mAmps at primary charge state S7 is useful for efficiently extracting the remaining about 50-60 percent of battery charge from AA-sized alkaline batteries. It should be appreciated that, in the illustrated embodiment, it is expected that Alkaline batteries will mostly operate in the medium- and low-current primary charge states S4, S7 because the properties of Alkaline batteries are more suited for longer durations of medium and/or low current discharges. The inventors have determined through testing that, on average, it takes about 90 seconds (total) to charge the supercapacitors 72 when transitions from S1 to S4 and from S4 to S7 are involved (as aggregated for both NiNM and Alkaline batteries), with individual charge times ranging from about 60 seconds to about 140 seconds when S7 is employed at any point in the charging process.

Transitions between Charge States:

The electronic hardware (e.g., charge circuit 80 and other device circuitry) was designed to be able to monitor various critical components and use those measurements to assist in the optimization of the supercapacitor charge cycle. In the illustrated embodiment, the battery voltage (Vbatt) and battery delta voltage (Vd) are monitored only during Initialization S0 and the primary charge states S1, S4 and S7; and the first boosted voltage (“5V2”) and second boosted voltage (“VDD_CAP”) are monitored through top-off charging S9 and beyond. The power source voltages (i.e. battery voltage (Vbatt) and battery delta voltage (Vd)) and the first and second boost regulator voltages (5V2 and VDD_CAP) are actively monitored along with the state and total charge times (t and T) to determine the battery type (i.e., NiNM or Alkaline) and their charge levels until the supercapacitor charge cycle is complete. These parameters are measured and employed to, among other things, determine the appropriate charge current to use with the battery type and battery charge level.

Battery Delta Voltage (Vd):

In the illustrated embodiment, battery delta voltage (Vd) is only measured in the high-current and medium-current primary charge states S1, S4. Preferably, the battery delta voltage (Vd) is only monitored for a short duration (at 100 ms intervals for a total of 1.6 seconds) when charging begins in each of S1 and S4. At initialization, a battery baseline voltage is recorded, and is used to compare against the active battery voltage (Vbatt) measurements for calculating the battery delta voltage (Vd) during primary charge states S1 and S4. This battery delta voltage (Vd) reading can be used to effectively determine how much charge remains in the batteries. As the battery charge level decreases, the battery delta voltage (Vd) increases with each load (e.g., S1 and S4). At a certain threshold, the battery delta voltage (Vd) can indicate that the batteries are not able to sustain the charge load, thereby requiring a transition to a lower current charge state (e.g., S1 to S4 and potentially S4 to S7). For the illustrated embodiment, the battery delta voltage (Vd) threshold of 0.6 Volt was chosen because it represents a response from the batteries that indicates they cannot sustain the applied load without collapsing the first boosted voltage (5V2). Thus, the algorithm 100 instructs the control unit 90 to transition to the lower primary charge state S4 or S7 before the 5V2 collapsing event should occur.

Battery Voltage (Vbatt):

The battery voltage (Vbatt) is monitored during Initialization S0, throughout the entirety of each primary charging state S1, S4, S7, and ceases as soon as the supercapacitors reach full charge. At each of these states, the control unit 90 samples the battery voltage (Vbatt) every 100 ms. During Initialization S0, in which the only load powered by charge current is the control unit voltage boost regulator 89 (3.3 Volts), if the battery voltage (Vbatt) measures at or below an initial threshold value (2.1 Volts), the charge current transitions directly to the Depleted Battery state S10. If the battery voltage (Vbatt) remains above the initial threshold value (2.1 Volts) during Initialization S0, and the first boosted voltage (5V2) measures at or above a respective threshold value (2.5 Volts), the charge current transitions to high-current primary charge state S1, at which supercapacitor charging commences, and sampling begins for S1. If the battery voltage (Vbatt) drops below a respective threshold value (1.9 Volts) during S1, the charge current transitions to S4. If the battery voltage (Vbatt) drops below a respective threshold value (1.9 Volts) during S4, the charge current transitions to S7. If the battery voltage (Vbatt) drops below a respective threshold value (1.6 Volts) during S7, the charge current transitions to S10.

A main reason that the battery voltage (Vbatt) is monitored during charge states S0, S1, S4, and S7 and employed to trigger state transitions is to operate as a safety measure for preventing collapse of the boosted voltage for operating the control unit 90 (3.3 Volts) during initialization and primary charging. Relatedly, monitoring the battery voltage (Vbatt) during charge states S0, S1, S4, and S7 also facilitates transitions to more conservative charging characteristics (e.g., lower charge currents) for battery types not well suited for high discharges. For example, when Alkaline batteries are employed, the battery voltage (Vbatt) at S1 measures promptly at or below the threshold value of 1.9 Volts, promptly transitioning the charge current to S4, which is better suited for Alkaline batteries. Stated differently, employing the battery voltage Vbatt as a transition parameter helps transition certain battery types to a more appropriate charge current (S4, S7), and to maximize the use of the remaining battery charge.

For the illustrated embodiment, one reason that 2.1 Volts was selected as the threshold battery voltage (Vbatt) during Initialization S0 is because 2.1 Volts was found to be sufficient to prevent the boosted voltage for operating the control unit 90 (3.3 Volts) from collapsing. One reason that 1.9 Volts was selected as the threshold battery voltage (Vbatt) during S1 and S4 is because 1.9 Volts is above the battery voltage (about 1.7 Volts) at which the first boosted voltage (5V2) was observed to collapse. Thus, employing 1.9 Volts as the threshold battery voltage (Vbatt) during S1 and S4 triggers a charge state transition before the first boosted voltage (5V2) collapsing event would occur. One reason that 1.6 Volts was selected as the threshold battery voltage (Vbatt) during S7 is because 1.6 Volts is above the minimum input voltage (about 0.5 Volts) necessary to have all electrical hardware of the device 2 operate as intended. Thus, employing 1.6 Volts as the threshold battery voltage (Vbatt) during S7 provides enough margin to indicate to the user with the power LED 25 that the batteries are depleted and need to be either replaced or recharged (e.g., via the USB-C port 67).

First Boosted Voltage (5V2):

The first boosted voltage (5V2) is the output of the first voltage boost regulator 88, which boosts the battery voltage and isolates the second voltage boost regulator 86 stage that creates the final voltage used to charge the supercapacitors 72. As the batteries lose charge, the power output capabilities generally decrease. The reduction of input power at the first voltage boost regulator 88 can cause the output thereof to collapse to under 2.0 Volts. The first boosted voltage (5V2) is monitored during Initialization S0, throughout the entirety of each primary charging state S1, S4, S7 and throughout top-off charging S9, and ceases after the supercapacitors reach full charge (e.g., after one or more impedance test pulses are delivered through the electrode(s) 10 to the tissue). For the illustrated example, at each of these states S0, S1, S4, S7, the first boosted voltage (5V2) is sampled at 100 ms intervals for at least 2.0 seconds (at least 20 samples), after which the samples will be continuously averaged. Thus, in the illustrated embodiment, the first boosted voltage (5V2) measurement is an average value based on at least 20 samples, which is thereafter continuously measured for continuous comparison against the voltage threshold (2.5V). After each state change between S0, S1, S4, and S7, the first boosted voltage (5V2) samples are reset and a new sample period begins until a subsequent state change.

The first boosted voltage (5V2) has a voltage threshold of a minimum of 2.5 Volts that was selected mainly as a safety feature to identify an occurrence of the collapse of the voltage necessary for supercapacitor charging. In particular, this safety feature provides a backup trigger condition for state transition, so that even when the battery delta voltage (Vd) and battery voltage (Vbatt) are adequate, if the first boosted voltage (5V2) collapsed to or below the threshold (2.5 Volts), the charge current will transition to a lower charge state. In particular, while in Initialization S0, if the first boosted voltage (5V2) measures at or below the threshold (2.5 Volts), the algorithm 100 instructs the control unit 90 to indicate a device error to the user. While at S1, if the first boosted voltage (5V2) measures at or below the threshold (2.5 Volts), the charge current will transition to S4. While at S4, if the first boosted voltage (5V2) measures at or below the threshold (2.5 Volts), the charge current will transition to S7. While at S7, if the first boosted voltage (5V2) measures at or below the threshold (2.5 Volts), the charge current will transition to the Depleted Battery state S10. For the illustrated embodiment, 2.5 Volts was selected as the threshold for the first boosted voltage (5V2) because 2.5 Volts provides a safety margin above the voltage to which the first voltage boost regulator 88 collapses (about 1.7 Volts), while being low enough to avoid giving false triggers due to noise or temporary dips in the 5V2 voltage output. It is also high enough that if the 5V2 output does collapse, it will ensure that it triggers the state change. When operating as intended, the first boosted voltage (5V2) remains steady at 5.2 Volts, but when it collapses, it has been observed to drop to about 1.7 Volts, therefore a threshold 2.5 V provides a sufficient margin to trigger a state transition before collapse.

It should also be appreciated that various features of the algorithm 100 can be adjusted or altered while remaining within the scope of the present disclosure. For example, one or more and up to each of the high-charge (2000 mAmps), medium-charge (446 mAmps), and low-charge (250 mAmps) currents can be adjusted to a higher or lower current magnitude to tailor the respective primary charge state(s) based on various factors. For example, such factors can include the use of battery configurations different from AA-sized NiMH batteries and Alkaline batteries. Additionally or alternatively, one or more an up to all of the threshold values for battery voltage (Vbatt), battery delta voltage (Vd), first boosted voltage (5V2), second boosted voltage (VDD_CAP), state charging duration (t), and total charging duration (T) can be adjusted based on various factors. Moreover, in other embodiments, the algorithm 100 optionally need not employ precisely three (3) primary charge states. For example, the algorithm 100 can optionally employ one (1), two (2), four (4), five (5), or more than five primary charge states, each having a preselected charge current. Additionally or alternatively, various features related to measurement sampling, such as sample quantity, sampling interval, and the timing of sampling commencement, etc., can be adjusted as needed.

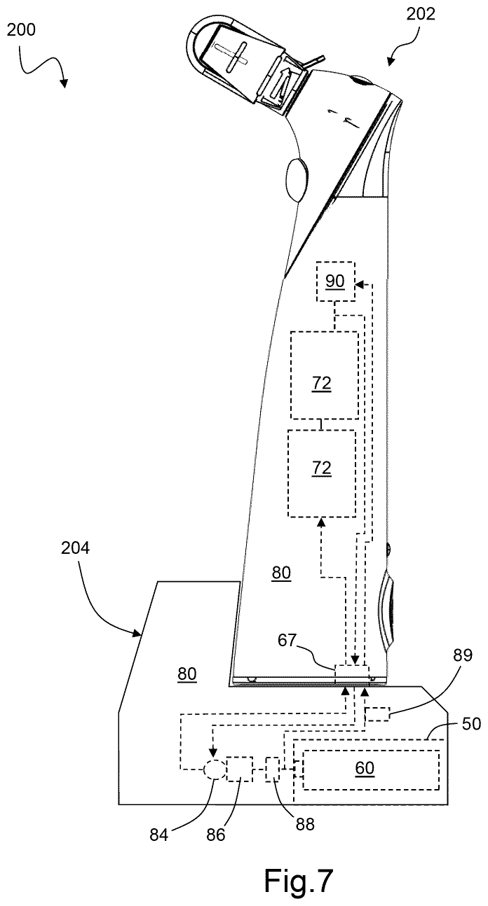

Referring now to FIG. 7, in additional embodiments, an example electroporation system 200 can include a handheld electroporation device 202 and a base station 204 configured to dock with the handheld electroporation device 202. In such embodiments, one or more components of the power supply unit 12 described above can be housed within the base station 204. For example, in the illustrated embodiment of the electroporation system 200, the base station 204 can house the battery unit 50, the batteries 60, the first and second voltage boost regulators 88, 86, the current regulation 84, and the control unit voltage boost regulator 89. In such embodiments, the second voltage boost regulator 86 can transmit the charge current to the supercapacitors 72 of the docked handset 202 via a charge port, such as the USB-C port 67 described above, or high-current DC contacts, and/or any electrical connection means that can handle the required energy transmission without degradation, by way of a non-limiting examples. Moreover, in such embodiments, the battery unit 50 in the base station 204 can be configured to hold additional batteries (e.g., more than two batteries 60) and/or more battery types, which, among other benefits, provides additional battery capacity and can enable the use of higher charge currents for charging the energy storage unit 70 (e.g., supercapacitors 72). Furthermore, in addition to housing batteries, the base station 204 can also be connectable to an external power adapter (e.g., for connection to a wall outlet or other power supply). These features can also further reduce the time necessary to charge the energy storage unit 70, and can also allow significantly more patients to be treated before recharging is required. Furthermore, by placing components of the charge circuit 80 within the base station 204, the handheld electroporation device 202 can be made even smaller and lighter, which can enhance usability. It should be appreciated that employing the base station 204 to house various components of the charge circuit 80 and/or for additional battery capacity can provide numerous other advantages. It should also be appreciated that in other embodiments, various components of the power supply unit 12 can be located in the handheld electroporation device 2 while others are located within the base station 204.

It should further be appreciated that, in embodiments employing a base station 204, various design modifications can be employed to ensure that the control unit 90 has adequate power to control operation of the handheld device 202. For example, one or more additional energy storage devices, such as one or more additional supercapacitor(s), capacitor(s), and/or battery(ies), can be located in the handheld device 202 for delivering power to the control unit 90, and which can be charged by the base station 204. It should further be appreciated that yet other modifications can be made to the configuration of the base station 204 and the associated handheld device 202 while remaining within the scope of the present disclosure.

It should be appreciated that the various features of the devices, systems, methods, and algorithms described above are provided as exemplary features for adapting the handheld electroporation devices to more efficiently charge and store power for delivering electroporation pulses. These parameters can be adjusted as needed without departing from the scope of the present disclosure.

It should further be appreciated when a numerical preposition (e.g., “first”, “second”, “third”) is used herein with reference to an element, component, dimension, process step, or a feature thereof, such numerical preposition is used to distinguish said element, component, dimension, and/or feature from another such element, component, dimension, process step, and/or feature, and is not to be limited to the specific numerical preposition used in that instance. For example, a “first” component can also be referred to as a “second” component in a different context without departing from the scope of the present disclosure, so long as said components (and/or elements, dimensions, process steps, and/or features) remain properly distinguished in the context in which the numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A method for preparing an electroporation device for delivering an electroporation treatment, comprising:
delivering a charge current from at least one battery through a charge circuit to a supercapacitor unit;
charging the supercapacitor unit with the charge current, wherein the charging step comprises:
measuring one or more input parameters of the charge current while the charge current is in at least one charge state of a plurality of charge states of charging the supercapacitor unit; and
at least one step of transitioning the charge current between charge states of the plurality of charge states responsive to the one or more measured input parameters, wherein transitioning the charge current comprises adjusting a magnitude of the charge current,
wherein the measuring and transitioning steps are automatically controlled by a control unit executing machine-readable instructions.

2. The method of claim 1, wherein at least one battery is selected from the group comprising Alkaline, NiMH, and Li-Ion batteries.

3. The method of claim 1, wherein at least one battery comprises a pair of batteries selected from the group comprising Alkaline and NiMH batteries.

4. The method of claim 1, wherein:
the one or more input parameters is selected from the group comprising: a battery voltage measured at a first location, a change in the battery voltage when a charge load is applied to the at least one battery, a first boosted voltage measured at a second location between the at least one battery and the supercapacitor unit, a second boosted voltage measured at a third location between the second location and the supercapacitor unit, and a charging duration, and
the method further comprises determining whether the one or more measured input parameters satisfies a condition statement for triggering the at least one transitioning step.

5. The method of claim 4, wherein:
the plurality of charge states comprises a set of primary charge states, and
when the transitioning step comprises transitioning the charge current from one charge state to another charge state that are both in the set of primary charge states, the adjusting step comprises decreasing the electric current.

6. The method of claim 5, wherein the set of primary charge states comprises:
a first primary charge state, in which the electric current is substantially maintained at a first current value, and
a second primary charge state, in which the electric current is substantially maintained at a second current value that is less than the first current value.

7. The method of claim 6, wherein the at least one transitioning step comprises transitioning the charge current from the first primary charge state to the second primary charge state, thereby adjusting the electric current from the first current value to the second current value.

8. The method of claim 7, wherein the first current value is in a range from about 1000 mAmps to about 3000 mAmps.

9. The method of claim 7, wherein the second current value is in a range from about 300 mAmps to about 600 mAmps.

10. The method of claim 5, further comprising boosting a voltage of the charge current from the battery voltage to the first boosted voltage, wherein the boosting step is performed by a first voltage boost regulator of the charge circuit, wherein the first boost regulator is located at or upstream of second location.

11. The method of claim 10, further comprising further boosting the voltage of the charge current from the first boosted voltage to the second boosted voltage, wherein the further boosting step is performed by a second voltage boost regulator of the charge circuit, wherein the second boost regulator is located either at the third location or intermediate the second and third locations.

12. The method of claim 11, wherein the battery voltage is in a range from about 0.5 volts to about 3.0 volts, the first boosted voltage is in a range from about 3.0 volts to about 7.0 volts, and the second boosted voltage is in a range from about 7.0 volts to about 20 volts.

13. The method of claim 11, wherein the measuring step comprises:
sampling the battery voltage at the first location at a sample interval; and
sampling the first boosted voltage at the second location at the sample interval.

14. The method of claim 13, wherein the sample interval is in a range from about 0.1 milliseconds to about 1.0 second.

15. The method of claim 13, wherein:
the control unit obtains the measured battery voltage samples and the measured first and second boosted voltage samples and calculates an average value of the first boosted voltage, and
the one or more input parameters include the battery voltage and the average value of the first boosted voltage.

16. The method of claim 6, wherein the at least one transitioning step comprises transitioning the charge current from one of the primary charge states to a top-off charge state after the supercapacitor unit reaches a full charge level.

17. The method of claim 16, further comprising, concurrent with the step of transitioning the charge current to the top-off charge state, communicating an indication signal indicating that the device is treatment ready.

18. The method of claim 16, wherein the supercapacitor unit has a voltage in a range of about 11.5 volts to about 12.5 volts when at the full charge level.

19. The method of claim 6, wherein:
the plurality of charge states includes an initial charge state, in which the measured one or more input parameters includes the battery voltage and no charge current is delivered to the supercapacitors, and
the at least one transitioning step comprises transitioning the charge current from the initial charge state to the first primary charge state,
the adjusting step comprises adjusting the charge current from an initial electric current value of substantially zero to the first current value, and
the method further comprises determining whether the measured battery voltage equals or exceeds a threshold voltage value prior to the at least one transitioning step.

20. The method of claim 6, wherein, in each of the primary charge states, the respective electric current value is maintained for at least a respective predetermined duration:
until either (1) the supercapacitor unit reaches the full charge level, or (2) the charging duration exceeds a respective duration limit associated with the respective primary charge state;
unless at least one of the one or more input parameters satisfies a threshold limit that triggers a transition to an abort state of the plurality of charge states before the supercapacitor unit reaches the full charge level or before the charging duration exceeds the respective duration limit.

21. The method of claim 20, wherein:
the at least one transitioning step comprises transitioning the charge current from one of the first and second primary charge states to the abort state responsive to (1) the charging duration exceeding the respective duration limit or (2) the control unit determines that the at least one of the one or more input parameters satisfies the threshold limit, and
the adjusting step comprises adjusting the respective electric current value substantially to zero.

22. The method of claim 7, wherein the set of primary charge states further comprises a third primary charge state, in which the magnitude of the charge current is substantially maintained at a third current value that is less than the second current value.

23. The method of claim 22, wherein the third current value is in a range from about 150 mAmps to about 300 mAmps.

24. The method of claim 22, wherein the at least one transitioning step further comprises transitioning the charge current from the second primary charge state to the third primary charge state, thereby adjusting the magnitude of the charge current from the second current value to the third current value.

25. The method of claim 24, wherein the at least one transitioning step further comprises transitioning the charge current from the third primary charge state to a top-off charge state after the supercapacitor unit reaches a full charge level.

26. The method of claim 22, wherein:
the one or more input parameters includes charging duration, and
the method further comprises:
maintaining the electric current at the third current value for at least a predetermined duration until either (1) the supercapacitor unit reaches the full charge level, or (2) the charging duration exceeds a duration limit associated with the third primary charge state,
determining that the charging duration exceeded the duration limit, and
responsively transitioning the charge current to an abort state of the plurality of charge states, thereby adjusting the electric current from the third electric current value substantially to zero.

27. The method of claim 1, further comprising communicating indication signals to a user, wherein each of the indication signals indicates a transition between charge states.

28. The method of claim 27, wherein the step of communicating indication signals comprises communicating optical indication signals to at least one LED display.

29. The method of claim 28, wherein optical indication signals are selected from the group comprising: a steady LED signal, a flashing LED signal, a first LED color, a second LED color, and a third LED color.

30. The method of claim 27, wherein the step of communicating indication signals comprises communicating at least one audible indication signal to a speaker carried by the electroporation device.

31. A method of using a handheld electroporation device, comprising:
delivering a charge current from at least one battery through a charge circuit to a supercapacitor unit, wherein the charge circuit and the supercapacitor unit are disposed within a device housing;
charging the supercapacitor unit with the charge current, wherein the charging step comprises:
measuring at least one voltage parameter of the at least one battery; and
adjusting a magnitude of the charge current to a current magnitude associated with a primary charge state responsive to the at least one measured voltage parameter,
wherein the measuring and transitioning steps are automatically controlled by a control unit executing machine-readable instructions; and
discharging an output signal from the supercapacitor unit after the supercapacitor unit is fully charged;
converting the output signal to one or more electroporation pulses; and
transmitting the one or more electroporation pulses to at least one electrode of the handheld electroporation device.

32. The method of claim 31, wherein the charging step comprises:
measuring the at least one voltage parameter while the charge current is in the primary charge state; and
determining whether the at least one measured voltage parameter has a measured value that satisfies a trigger condition of a condition statement while the charge current is in the primary charge state.

33. The method of claim 32, wherein that charging step further comprises:
determining that the measured value satisfies the trigger condition while the charge current is in the primary charge state; and
responsively reducing the magnitude of the charge current to a second current magnitude associated with a second primary charge state.

34. The method of claim 33, wherein the charging step comprises:
measuring the charge level of the supercapacitor unit; and
measuring the charging duration in which the charge current is in the respective primary charge state.

35. The method of claim 34, wherein the charging step comprises:
determining that the charge level of the supercapacitor unit is fully charged; and
transitioning the charge current to a top-off charge state.

36. The method of claim 35, wherein the charging step further comprises:
determining whether the charging duration exceeds a threshold duration before transitioning the charge current to the top-off charge state; and
while the charge current is in the top-off charge state, either:
communicating a first indication signal to the user if the charging duration exceeded the threshold duration, or
communicating a second, different indication signal to the user if the charging duration was less than the threshold duration.

37. The method of claim 34, further comprising:
terminating the charging step if the charging duration within the respective primary charge state exceeds a second threshold duration that is greater than the threshold duration; and
communicating a third indication signal to the user indicating that the charging step is terminated.

38. The method of claim 34, further comprising:
disconnecting the at least one battery from the battery unit;
connecting at least one second battery to the battery unit; and
repeating the delivering and charging steps with the at least one second battery.

39. The method of claim 31, wherein the at least one voltage parameter comprises battery voltage, and the measuring step comprises sampling the battery voltage at regular sample intervals during the charging step.

40. The method of claim 39, wherein battery voltage is a first voltage parameter of the at least one voltage parameter, and the at least one voltage parameter further comprises a second voltage parameter, wherein the second voltage parameter comprises battery delta voltage, and the measuring step comprises sampling the battery delta voltage at the regular sample intervals during the charging step.

41. A power supply unit for an electroporation device, comprising:
a battery unit configured to connect interchangeably to a first type of battery and a second type of battery;
an energy storage unit that is configured to be charged interchangeably by the first type of battery and the second type of battery, wherein the energy storage unit is further configured to discharge a total energy of at least about 30 Joules;
an integrated circuit configured to execute computer readable instructions; and
a charge circuit in electrical communication with the battery unit and the energy storage unit, wherein the charge circuit is operable under control of the integrated circuit such that:
the charge circuit is configured to measure one or more input parameters of a charge current drawn from the battery unit, and
the charge circuit is further configured to adjust a magnitude of the charge current responsive to the measured one or more input parameters while charging the energy storage unit with the charge current.

42. The power supply unit of claim 41, wherein the first and second types of batteries are selected from the group comprising alkaline, NiMH, and lithium-ion.

43. The power supply unit of claim 42, wherein the first type of battery is alkaline and the second type of battery is NiMH.

44. The power supply unit of claim 43, wherein the battery unit is configured to connect to a pair of alkaline batteries interchangeably with a pair of NiNM batteries, and the battery unit connects each respective pair of batteries in series.

45. The power supply unit of claim 42, wherein the integrated circuit is configured to estimate an initial charge level of the respective first type of battery or the second type of battery connected to the battery unit, and the integrated circuit is further configured to reduce the magnitude of the charge current responsive to the estimated initial charge level.

46. The power supply unit of claim 41, wherein the energy storage unit comprises a plurality of supercapacitors connected in series.

47. The power supply unit of claim 41, wherein the charge circuit includes at least one sample location at which at least one of the one or more input parameters are measured, and the at least one sample location is in electronic communication with the integrated circuit.

48. The power supply unit of claim 47, wherein the charge circuit comprises at least one voltage boost regulator between the battery unit and the energy storage unit, wherein the at least one voltage boost regulator is configured to boost a voltage of the charge current to a boosted voltage.

49. The power supply unit of claim 48, wherein the at least one voltage boost regulator comprises:
a first voltage boost regulator at a first location in the charge circuit, wherein the first voltage boost regulator is configured to boost the voltage of the charge current from an initial battery voltage to a first boosted voltage; and
a second voltage boost regulator at a second location in the charge circuit, wherein the second location is between the first location and the energy storage unit, and the second voltage boost regulator is configured to boost the voltage of the charge current from the first boosted voltage to a second boosted voltage, wherein the second boosted voltage is substantially equivalent to a charge voltage at which the energy storage unit is charged.

50. The power supply unit of claim 49, wherein the first boosted voltage is in a range of about 2.0 volts to about 10.0 volts, and the second boosted voltage is in a range from about 5.0 volts to about 20 volts.

51. The power supply unit of claim 49, wherein the charge circuit comprises:
a first sample location between the battery unit and the first voltage boost regulator, wherein the initial battery voltage is measured at the first sample location; and
a second sample location between the first voltage boost regulator and the second voltage boost regulator, wherein the first boosted voltage is measured at the second sample location,
wherein the first and second sample locations are in electronic communication with the integrated circuit.

52. The power supply unit of claim 41, wherein the power supply unit is insertable within a device housing of the electroporation device.

53. The power supply unit of claim 52, wherein the battery unit comprises a battery housing that is insertable at least partially within the device housing, the battery housing having at least one battery receptacle configured to receive, interchangeably, the first type of battery and the second type of battery.

54. The power supply unit of claim 53, wherein the at least one battery receptacle comprises a first battery receptacle and a second battery receptacle, wherein the first and second battery receptacles are each configured to receive a pair of the first type of battery interchangeably with a pair of the second type of battery.

55. The power supply unit of claim 53, further comprising at least one indicator device for indicating a charge status of the energy storage unit.

56. The power supply unit of claim 55, wherein the at least one indicator device comprises an LED display for communicating one or more optical signals regarding the charge status of the energy storage unit, and the LED display is receivable within an aperture of the device housing.

57. The power supply unit of claim 56, wherein the at least one indicator device comprises a second LED display for communicating one or more optical signals indicating when the energy storage unit is fully charged.

58. The power supply unit of claim 53, wherein the charge circuit is disposed on a circuit board insertable within the device housing.

59. The power supply unit of claim 58, wherein the energy storage unit is mountable to the circuit board, and the battery housing is connectable to the circuit board, wherein the battery housing has at least one electrical contact connectable with at least one electrical contact of the circuit board for providing electronic communication between the battery unit and the charge circuit.

60. The power supply unit of claim 53, wherein the battery housing includes an electronic communication port for receiving an electronic communication device.

61. The power supply unit of claim 60, wherein:
the electronic communication port is a USB-C port, and
the battery housing carries an LED display for indicating when the USB-C port is coupled to a USB-C cable.

62. The power supply unit of claim 41, wherein the energy storage unit is configured to discharge a total energy of about 90 Joules.

63. The power supply unit of claim 41, wherein the energy storage unit is configured to discharge discrete portions of the total energy, and the discrete portions are convertible into one or more electroporation pulses.

64. A handheld electroporation device, comprising:
a device housing;
at least one electrode connectable to the device housing;
a battery unit at least partially insertable within the device housing, the battery unit configured to connect interchangeably to a first type of battery and a second type of battery;
a supercapacitor unit that is configured to be charged interchangeably by the first type of battery and the second type of battery, wherein the supercapacitor unit is further configured to discharge a total energy of at least about 30 Joules;
a charge circuit in electrical communication with the battery unit and the supercapacitor unit, the charge circuit configured to deliver a charge current from the battery unit to the supercapacitor unit for charging the supercapacitor unit,
wherein, under control of an integrated circuit configured to execute machine readable instructions, the charge circuit is configured to measure at least one voltage parameter of the respective first type of battery or second type of battery and to responsively adjust a magnitude of charge current to thereby reduce a rate at which the respective first type of battery or second type of battery loses charge while charging the supercapacitor unit.

65. The handheld electroporation device of claim 64, wherein the at least one voltage parameter comprises battery voltage.

66. The handheld electroporation device of claim 64, wherein the at least one voltage parameter further comprises battery delta voltage, measured as a difference between:
the battery voltage when no load is applied thereto, and
the battery voltage when the supercapacitor unit is charged by the battery unit.

67. The handheld electroporation device of claim 64, wherein the energy storage unit is configured to discharge a total energy of about 90 Joules.

68. The handheld electroporation device of claim 64, wherein the energy storage unit is configured to discharge discrete portions of the total energy.

69. The handheld electroporation device of claim 68, further comprising additional circuitry for converting the discrete portions of the total energy into one or more electroporation pulses.

70. The handheld electroporation device of claim 69, wherein the additional circuitry is further configured to deliver the one or more electroporation pulses to the at least one electrode.
